(19) United States
(12) Patent Application Publication
(10) Pub. No.: US 2018/0049856 A1
(43) Pub. Date: Feb. 22, 2018

(54) METHOD AND SYSTEM FOR DELIVERY OF PARTICLES IN A ROOT CANAL SYSTEM

(71) Applicants: Dan Andrei Ionescu, Oakville (CA); Anil Kishen, Toronto (CA)

(72) Inventors: Dan Andrei Ionescu, Oakville (CA); Anil Kishen, Toronto (CA)

(21) Appl. No.: 15/549,860

(22) PCT Filed: Feb. 9, 2016
(86) PCT No.: PCT/CA2016/050117

§ 371 Date: Aug. 9, 2017

(51) Int. Cl. A61C 19/06; A61C 5/40; A61C 5/82; A61N 1/04; A61N 1/30

(52) U.S. Cl. CPC A61C 19/06; A61C 5/40; A61N 1/306; A61N 1/044; A61N 1/0472; A61C 5/82

(57) ABSTRACT

Various embodiments are described herein for a system and method for reducing bacteria of a tooth receiving root canal treatment, the tooth having a root canal, and the method includes deploying an intracanal electrode within the root canal; deploying an intraoral electrode in close proximity to the root canal and external to the tooth; and applying a potential difference between the electrodes to create an electric field therebetween to reduce the bacteria of the tooth.

### CROSS-REFERENCE TO RELATED APPLICATION

This application claims the benefit of United States Provisional Patent Application No. 62/113,524, filed Feb. 9, 2015; the entire contents of Patent Application No. 62/113,524 are hereby incorporated by reference.

### FIELD

The various example embodiments described herein relate to a system and method for endodontic delivery of particles into a root canal system and via root canals to the external root surface of a tooth.

### BACKGROUND

The involvement of biofilms in endodontic infections has been well recognized and has gained much attention in recent years. Studies of endodontic microbiota in primary and persistent infections have demonstrated that bacterial colonization of the root canal system occurs mostly as polymicrobial aggregations, adherent to dentinal surfaces, and embedded in an extracellular polymeric substance (EPS), termed as biofilms [1-3]. The EPS, with its highly charged and interwoven structure, deters penetration of antimicrobials by acting as a physical barrier and neutralizer for biochemical reactions [4]. Such organization into communities of microorganisms has been shown to provide ecological advantages and offer protection from host responses as well as antimicrobials [5, 6].

Multiple scanning and transmission electron microscopy studies (SEM, TEM) have shown that necrotic and infected root canal systems may be coated with a bacterial plaque extending into canal intricacies [1, 3], dentinal tubules [7] and on certain occasions extending externally onto the apical portion of the cementum [8]. Bacterial colonies outside of the root canal system have been described as forming biofilm-like structures on the cementum adjacent to the apical foramen [2, 8, 9] or as forming cohesive colonies within the periapical lesion [10, 11]. It is known that persistent/refractory apical periodontitis may be induced by bacterial biofilms on the external root surface that may evade host defense mechanisms while remaining unreachable by endodontic irrigants [12-14]. Moreover, recent studies utilizing advanced molecular techniques together with imaging by electron and light microscopy provide evidence that such extraradicular biofilms are not uncommon, especially in cases that do not respond to conventional endodontic treatment [15-18].

A number of studies have suggested that in some cases, persistent apical periodontitis may be caused by bacterial biofilms on the external root surface that can evade host defense mechanisms and endodontic irrigants, while continuing to exhibit pathogenic effects [13, 14]. An apical surgery procedure may be carried out in cases where conventional endodontic therapy is not effective, whereby an incision is made in the gingival tissues, a flap of that tissue is elevated, an osteotomy is made through the alveolar bone and the apex of the root is amputated [22]. A root end preparation and tight filling may be carried out with microsurgical ultrasonic instruments [23].

The goal of endodontic therapy is to eliminate bacteria and prevent apical periodontitis and to preserve teeth functionality without prejudice to a person's health [28, 29]. Root canal therapy may be performed on teeth with irreversibly inflamed dental pulps, in an attempt to prevent apical periodontitis, or on teeth with apical periodontitis aiming to treat infection. The condition in which the root canal presents may therefore vary from that of an intact pulp-dentin complex, through a partially degraded pulp with areas of infection, to a system completely coated with mature bacterial biofilm [1]. Complete elimination of bacteria from infected root canals while maintaining the structural integrity of dentin in endodontically treated teeth has proven to be a daunting task in dentistry [19, 20]. The incomplete elimination of microbes may be attributed to the tenacity of bacteria in biofilm form, and localization of biofilm in root canal complexities or on extraradicular surfaces.

Current non-surgical endodontic disinfection strategies are confined to the root canal system and focus predominantly on mechanical and chemical debridement of the internal contents. Mechanical debridement may be carried out by enlarging the root canal space, sometimes requiring the sacrifice of solid dentin. Such a procedure may lead to extensive loss of tooth structure and overall weakening of the tooth. Chemical debridement techniques similarly suffer from limitations posed by bacteria organized in biofilms, anatomical complexities of the root canal system, and the toxicity of chemical disinfectants.

### SUMMARY OF VARIOUS EMBODIMENTS

The various example embodiments described herein relate to a system and method for endodontic delivery of particles to a root canal system and external to the root canal system (i.e. external root surface and periapical tissues) through the portals of exit in a root canal. More particularly, the various embodiments relate to a system and method for transporting charged (cationic or anionic) particles in suspension through the root canal anatomy and depositing the particles at a target site within the tooth, root surface or the periapical tissues. The particles may have a narrow size distribution or a wide size distribution. The particles may have a large size such as powders, or may have a small size on the order or microns or nanometers.

In a broad aspect, at least one example embodiment provides a method of utilizing electrophoretic currents for the delivery of biocompatible, charged substances to the periapical tissues in close proximity of the apical foramen which may result in one or more of the disruption of biofilms, the facilitation of healing, the promotion of remineralisation, as well as the delivery and deposition of bioactive and antibacterial substances.

In a broad aspect, at least one embodiment described herein provides a method of reducing bacteria of a tooth receiving root canal treatment, the tooth having a root canal, wherein the method comprises: deploying an intracanal electrode within the root canal; deploying an intraoral electrode in close proximity to the root canal and external to the tooth; and applying a potential difference between the electrodes to create an electric field therebetween to reduce the bacteria of the tooth.

In at least some embodiments, the method may further comprise introducing a carrier solution having charged particles into the root canal and applying the potential difference to create the electric field to induce a flow of the charged particles to structures within and adjacent to the tooth.

In at least some embodiments, the act of deploying the intraoral electrode may comprise placing the intraoral electrode in direct contact with soft tissue surrounding the tooth, placing the intraoral electrode in direct contact with tooth enamel or placing the intraoral electrode in direct contact with periodontal ligament space.

In at least some embodiments, the intraoral electrode may comprise a conductive ring and the method may comprise placing the intraoral electrode in contact with one or more areas of soft oral tissue adjacent to the tooth.

In at least some embodiments, the intraoral electrode may comprise a microneedle electrode and the method may comprise deploying the microneedle electrode on soft oral tissue adjacent to the tooth.

In at least some embodiments, the intraoral electrode may comprise a modified rubber dam clamp that is conductive and the method may comprise placing the modified rubber dam clamp adjacent to or penetrating into the gingival tissues on a buccal and a lingual region of the tooth.

In at least some embodiments, the method may comprise applying a potential difference between the electrodes of at least 20V to 70V DC or AC and more preferably about 50V DC or AC.

In at least some embodiments, the method may comprise applying the potential difference between the electrodes to provide the electric field with a field strength of about 15 V/cm to about 45 V/cm.

In at least some embodiments, the method may further comprise delivering the charged particles to locations corresponding to at least one of an external root surface, periapical region, and cementum surface of the root canal.

In at least some embodiments, the method may further comprise at least some of the particles forming a coating of particle film on the at least one of the external root surface, periapical region, and cementum surface of the root canal.

In at least some embodiments, the method may comprise applying the electric field to accumulate at least some of the charged particles in a region of the tooth corresponding to the apical delta.

In at least some embodiments, the method may comprise applying the electric field to form a film of at least some of the charged particles on an extraradicular root surface of the tooth.

In at least some embodiments, the method may further comprise providing the carrier solution with charged particles having at least one of a sufficiently low conductivity of about 5-500 μS/m, a low viscosity of about 0.5-5 cP and a high dielectric constant of about 50-80 to facilitate movement and deposition of charged particles within the tooth.

In at least some embodiments, the charged particles may comprise nanoparticles with a diameter less than 1 μm and belong to a class of particles that are sensitive to the application of an electric field.

In at least some embodiments, the charged particles may comprise chitosan nanoparticles.

In a broad aspect, at least one embodiment described herein provides a system for reducing bacteria of a tooth receiving root canal treatment, the tooth having a root canal, wherein the system comprises an intracanal electrode that is deployed within the root canal; an intraoral electrode that is deployed in close proximity to the tooth canal but external of the tooth; and a signal generator coupled to the electrodes for applying a potential difference between the electrodes to create an electric field therebetween to reduce the bacteria of the tooth.

In at least some embodiments, the system may further comprise a carrier solution having charged particles and the charged particles are introduced into the root canal before or during application of the electric field wherein the electric field is created to induce a flow of the charged particles to structures within and adjacent to the tooth.

In at least some embodiments, the system may further comprise a power supply that is coupled to the signal generator and is configured to maintain at least one of a constant voltage and a constant current output.

In at least some embodiments, the intracanal electrode may comprise a modified conducting endodontic file having an electrically conducting shaft and an insulated tip that is configured to laterally drive the charged particles towards dentin of the tooth to create an Electrophoretic Deposition (EPD) on the dentinal walls of the tooth during use. Optionally, in some embodiments, a portion of the shaft is insulated to prevent aberrant currents in cervical portions of the tooth and adjacent teeth.

In at least some embodiments, the intracanal electrode may comprise a modified conducting endodontic file have an electrically insulated shaft and a conducting tip to generate electric field lines through an apical foramen or other ramifications of the tooth during use.

In at least some embodiments, the intracanal electrode may comprise a modified conductive ultrasonic tip, at least a portion of the intracanal electrode is covered by an insulating sheath and ultrasonic energy is delivered before, during or after application of the electric field.

In at least some embodiments, the intracanal electrode may comprises a hollow needle that is coupled to a fluid reservoir for providing the carrier solution with the charged particles during use, at least a portion of the needle is insulated to direct the electrical field directed toward lateral or apical aspects of the root canal.

In at least some embodiments, the needle may be open-ended, side-beveled, or side-vented for delivering and replenishing the carrier solution into the root canal simultaneously or subsequently to delivering electric current within the root canal.

In at least some embodiments, the intracanal electrode may be connectable to a dental ultrasonic unit.

In at least some embodiments, the intraoral electrode may be configured and deployed to make direct contact with one of soft tissue surrounding the tooth, tooth enamel of the tooth, and a periodontal ligament space of the tooth during use.

In at least some embodiments, the intraoral electrode may comprise a conductive ring that is configured to be placed around the tooth and make contact with one or more areas of soft oral tissue around the tooth during use.

In at least some embodiments, the intraoral electrode may comprise a microneedle array electrode that is configured for deployment on the soft oral tissue adjacent the tooth during use.

In at least some embodiments, the intraoral electrode may comprise a modified rubber dam clamp that is conductive and is configured to come into contact or penetrate gingival tissues on a buccal region and a lingual region of the tooth.

In at least some embodiments, potential difference generated by the signal generator between the electrodes may be at least 20 V to 70 V DC or AC and more preferably about 50V DC or AC.

In at least some embodiments, the potential generated by the signal generator may produce the electric field between the electrodes with an electric field strength from about 15 V/cm to about 45 V/cm.

In at least some embodiments, the potential difference may be applied from 1 to 15 minutes and in some cases may be about 10 minutes.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a block diagram of an example embodiment of a system for delivering particles to a root canal system in accordance with the teachings herein.

FIGS. 2A-2C show side, cross-sectional and top views, respectively, of a schematic representation of an example embodiment of an intraoral electrode arrangement in accordance with the teachings herein.

FIG. 3A-3D show various example embodiments of an intracanal electrode in accordance with the teachings herein.

FIG. 3E is a diagram illustrating a deployed intraoral electrode and a deployed intracanal electrode in accordance with the teachings herein.

FIGS. 3F-3G are diagrams illustrating deployment of alternative types of example embodiments of intracanal electrodes in accordance with the teachings herein.

FIGS. 3H-3J are diagrams illustrating deployment of alternative types of example embodiments of intraoral electrodes in accordance with the teachings herein.

FIG. 4A is a Computer Aided Design (CAD) drawing based on the morphology of a maxillary central incisor with a lateral canal and a periapical lesion utilized for computer simulation and microfluidic device fabrication.

FIGS. 4B and 4C show example simulation outcomes for the generation of electric fields, current densities and particle trajectories in accordance with the teachings herein.

FIG. 5A is a photomicrograph of a microfluidic device that incorporates the simulation model of FIG. 4A.

FIGS. 5B-5E are photomicrographs demonstrating particle distribution within the device upon application of an electric field in accordance with the teachings herein.

FIG. 5F is a diagram illustrating the locations of the microfluidic device from which the images of FIGS. 5A-5E are obtained.

FIGS. 6A and 6B are plots illustrating the antimicrobial effects of an applied electric current in the absence and in the presence of any antimicrobial particles, respectively, on planktonic bacteria as a function of time in accordance with the teachings herein.

FIGS. 7A-7C show fluorescence emission characteristics of biofilms with and without treatment in accordance with the teachings herein.

FIGS. 8A-8B show graphs indicating the mechanical properties of root dentin with and without treatment with nanoparticles in accordance with the teachings herein.

FIGS. 9A-9C show scanning electron micrographs of surfaces of the root canal and external root surface coated with chitosan nanoparticles in accordance with the teachings herein.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to systems and methods having all of the features of any one of the described systems or methods described below or to features common to multiple or all of various embodiments described herein. It is possible that there may be a system or method described herein that is not an embodiment of any claimed subject matter. Any subject matter that is described herein that is not claimed in this document may be the subject matter of another protective instrument, for example, a continuing patent application, and the applicants, inventors or owners do not intend to abandon, disclaim or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that, as used herein, the wording “and/or” is intended to represent an inclusive-or. That is, “X and/or Y” is intended to mean X or Y or both X and Y, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Conditions such as apical periodontitis are biofilm-induced diseases that may involve both the root canal system (where they may form intracanal bacterial biofilms) and the extraradicular surfaces (where they may form extraradicular bacterial biofilms). Conventional endodontic treatment methods are limited, in part, by a clinician's ability to eradicate biofilms located within difficult-to-access areas of the root canal system and dentinal tubules, as well as biofilms established on the extraradicular surface. It is generally believed that the major cause of root canal failure is the persistence of microorganisms in the apical part of root-filled teeth, the dentinal tubules and the periapical surface of the root. Furthermore, long-term outcomes of teeth treated by root canals may be compromised by aggressive instrumentation and removal of sound tooth structure, which predispose the tooth crown and root to fracture.

Despite many technological advances, the reported success rates for both primary and secondary endodontic treatments have not improved over the last four or five decades [24-26]. It is generally believed that the major cause of root canal failure is the persistence of microorganisms associated with root-filled teeth. In accordance with the teachings herein, the inventors have realized that an inherent plateau in endodontic clinical outcomes may be in part due to the inability to address bacteria present in the apical intricacies of the root canal or on the external root surface during the initial endodontic therapy or retreatment.

Current non-surgical endodontic disinfection strategies are generally confined to the root canal system and focus predominantly on chemical and mechanical debridement of the internal contents (including, but not limited to, pulp tissue, necrotic remnants, bacteria, toxins, byproducts of infection, and dentinal debris). As mentioned previously, these treatment strategies face several challenges posed by bacteria organized in biofilms, anatomical complexities of the root canal system, and factors associated with the toxicity of chemical disinfectants. Furthermore, current non-surgical techniques do not attempt to eradicate bacteria located in the periapical region and on the external root surface.

It is desirable to overcome the current limitations and drawbacks of conventional endodontic treatment with the aim of disrupting the biofilm structure while simultaneously killing the resident bacteria even in locations that may not be accessible by conventional root canal instrumentation and irrigation procedures. One such approach, in accordance with the teachings herein, is to provide a system and method of introducing, transporting and depositing materials into the root canal for purposes including, but not limited to, at least one of disinfection, medicinal, anti-inflammatory, analgesic, regenerative, re-mineralization, root filling and biological healing by tissue regeneration/organized repair. For example, in at least one example embodiment described in accordance with the teachings herein, antimicrobial nanoparticles may be used for root canal disinfection while taking advantage of their unique physical and chemical characteristics.

In recent years, a significant body of research has developed, which examines electrical methods of controlling the growth of microorganisms. It is known that an electrical current can impart antimicrobial effects. For example, as early as 1919, it was reported that sterilization of milk could be achieved using an alternating current (AC), a process now known as “ohmic heating” [54]. Since that time various other scientific studies have demonstrated that electric fields, both AC and DC, may also disrupt bacterial integrity and possess quantifiable antibacterial properties [42, 43]. These findings have led to the development of several techniques for controlling bacterial adhesion and biofilm formation on various device surfaces. Little attempt has been made to utilize these techniques in the field of medicine or dentistry or on a human patients. The direct killing of bacteria by electric current has been referred to as the “electricidal effect”. It is also well understood that the concentration of antibiotics and biocides required to kill bacteria residing within a biofilm matrix can be 500-5000 times greater than those needed to kill planktonic cells (e.g. cells in suspension) of the same species [57-59]. It has been shown that the application of an electric current can reduce the very high concentrations of certain antimicrobials needed to kill biofilm bacteria to levels close to those needed to kill planktonic bacteria of the same species [53]. Therefore, it may be desirable to consider application of electric currents in conjunction with an antimicrobial agent so as to augment the antimicrobial effects against certain bacteria in biofilms, a concept which has been referred to in the field of engineering as the “bioelectric effect”.

### Electrokinetic Transport Phenomena

The electrokinetic phenomena describe a family of effects that occur in heterogeneous fluids, or fluids containing particles, under the influence of an electric field. These effects include electroosmosis, electrophoresis, streaming potential and sedimentation potential [45, 46]. These phenomena may arise as a result of the interactions between electric charges and liquids, and are often characterized by the presence of an electrical double layer, as described in more detail below.

Of interest in the present disclosure is the application of the electrophoretic and electroosmotic principles for endodontic treatment. In particular, the use of electrophoretic currents for the delivery of biocompatible, bioactive, charged substances to difficult-to-access areas of the root canal system is described herein. Specifically, the various embodiments described herein are directed to a system and method for disrupting bacterial biofilms, facilitating healing, and promoting re-mineralization of dentin with the use of electric currents. Therefore, it may be appreciated that delivery of antimicrobial particles in accordance with the teachings described herein may provide an effective approach to the disinfection of the root canal and surrounding areas. Additionally, the reduced invasiveness relative to conventional root canal procedures may preserve structural components such as dentin thereby improving the overall mechanical properties of the tooth being treated. Additionally, deposition of the antimicrobial particles into the tooth may prevent subsequent bacterial recolonization, repair dentinal cracks, and block portals of exit by stimulating hard tissue formation at the apex of the tooth.

The process of electrophoresis generally refers to the transport of charged colloidal particles under the influence of an electric field. When subjected to a non-uniform electrostatic field, particles in suspension may move at characteristic velocities determined by their charges, size and mobility [40, 41]. These phenomena are of relevance in many medical fields for manipulation of biological materials including, but not limited to, proteins, enzymes, cells, colloids, polymers and solid inorganic particles [43]. The subsequent ordered deposition of the particulate matter on a surface towards which it is attracted is termed electrophoretic deposition (EPD). Due to its simple and cost effective nature, EPD may be used for the fabrication of thin films and coatings of organic, inorganic and composite materials on substrates of complex shapes and surface areas [43]. These concepts may be adapted herein for the generation of films, comprising antimicrobial particles for example, on the surfaces of the root canal, dentinal tubules and other dental anatomies.

Particles in suspension may exhibit a quantity known as the zeta potential, or surface charge. The presence of a net charge may affect the distribution of ions surrounding the surface of a particle, for example, resulting in an increase in the concentration of counter-ions immediately adjacent to this surface. The region over which this influence extends is called the electrical double layer (EDL). First identified by Helmholtz in 1879, this layer is thought to comprise of two separate regions: an inner layer with strongly bound ions (stern layer) and on outer layer with loosely associated ions (diffuse layer). As the particle moves through a liquid solution due to an applied electric field or gravity, the bound ions may move along with it. At some distance from the particle there may exist a “boundary” beyond which ions do not move along with the particle. This distance, known as the Debye length (λ_{D}), may be located somewhere within the “diffuse layer” and demarcates the surface of hydrodynamic shear, or the slipping plane [46]. A similar electrical double layer develops adjacent to any solid phase in contact with an aqueous medium [46]. As such, an EDL with a characteristic Debye length may develop adjacent to stationary objects with charged surfaces, such as the wall of a microfluidic system.

When an electric field is applied, the charged particle may migrate toward the anode or cathode depending on the polarity of the EDL. A charged ion cloud made up of counter-ions may form as a result of their attraction to the particle. As the particle migrates through the liquid, counter-ions may move in and out of its charged ion cloud, or EDL. This exchange of counter-ions is responsible for electrophoretic transport and electroosmotic flow. The electrophoretic velocity (vₑₚ) is proportional to the magnitude of the electric field (E) and the particle's electrophoretic mobility (μₑₚ). This relationship can be explained utilizing Hückel's approximation for thick double layers and small particles per equation 1,

$${v}_{ep} = {μ}_{ep} E = \frac{{ɛɛ}_{0} {ζ}_{np}}{1.5 η} E \qquad ( 1 )$$

where ∈ is the dielectric constant of the solution, ∈₀ is the permittivity of free space, ζₙₚ is the zeta potential of the particle and η represents viscosity of the solvent [69].

Applying a voltage potential across a conductive substrate may further direct positively charged ions to move in the direction of the negative electrode, since, as the ions move, they may draw water molecules along with them. This phenomenon may be termed “electro-osmotic” flow and may particularly be notable in channels of narrow diameter (e.g. in the micrometer range). Note, however, that as the diameter of the channels narrow further (e.g. in the sub-micrometer range), influence from the surface charge of said channel may become more relevant and may need to be taken into consideration. The velocity due to electro-osmosis (vₑₒ) can be determined by the Helmholtz-Smoluchowski equation according to equation (2):

$${v}_{eo} = {μ}_{eo} E = \frac{{ɛɛ}_{0} {ζ}_{c}}{η} E \qquad ( 2 )$$

where μₑₒ is the electroosmotic mobility and ζ_{c} is the zeta potential of the microchannel. The resultant combined effects of electrophoresis and electro-osmosis may enable particle transport and deposition.

A particle's total velocity (vₜₒₜ) as it moves through a microfluidic system is the sum of its electrophoretic velocity and the electroosmotic flow velocity, as shown in equation 3.

vₜₒₜ=vₑₒ+vₑₚ (3)

In an EPD process, it is generally preferred that all of the applied electric field should be utilized to effect particle electrophoresis, the driving force for EPD. In reality an EPD suspension is far from ideal due to the presence of free ions in addition to the particles in suspension. A portion of the current may be carried not only by the charged particles but also by the free ions co-existing in the suspension. The actual particle movement in a liquid media depends not only on the value of the current density passing through the suspension but also on the geometry of the electrophoresis cell and the physical and chemical parameters of the system. Practically speaking a number of parameters, discussed in more detail below, influence EPD which fall in two groups; (1) suspension parameters including dielectric constant and carrier liquid viscosity, and (2) process parameters such as the electric field strength and the duration of time in which the electric field is engaged [48].

Candidate substances that may be used in the suspension can include, but are not limited to, antibacterial substances, antibiotics, nanoparticles, sealing materials, re-mineralization solutions, and root canal fillers. Of interest for endodontic disinfection is the use of nanoparticles, which are known to exhibit antimicrobial activities as a result of their polycationic nature in combination with higher surface area and charge density. The method of action which results in an antimicrobial effect relies on an entirely different mechanism as compared to traditional antibiotics. For example, the size and charge density of nanoparticles may provide a greater electrostatic force and a more intimate interaction with the negatively charged bacteria cell [27]. The accumulation of a large number of nanoparticles on the cell membrane of a bacterium may lead to increased membrane permeability and deterioration of membrane function [30, 31]. The inventors have found that this physical interaction suggests that cationic nanoparticles may provide broad spectrum antibacterial properties and may decrease the potential for the development of bacterial resistance.

Nanoparticles ranging from 1 nm to 100 nm (i.e. less than 1 μm) in diameter may be fabricated using any number of materials including carbon, semiconductor, metal oxides and hydrocarbons. For endodontic use, it may be desirable to use nanoparticles known to be stable and biocompatible (i.e. low toxicity). For example, chitosan nanoparticles (CSnp) may be appropriate for endodontic treatment because of its known stability and safety [27, 32-36]. Chitosan is a natural nontoxic biopolymer produced by the deacetylation of chitin, a major component of the shells of crustaceans such as crab, shrimp, and crawfish. CSnp is known to possess antibacterial [27] and antibiofilm [32] properties and may be able to disrupt multilayered, three-dimensional bacterial biofilm structures in vitro [32]. Furthermore this natural bioactive biopolymer has been well characterized and is known to exhibit thin film-forming characteristics. The intrinsic positive charge of CSnp may be attractive for electrophoretic transport and electrophoretic deposition (EPD). Studies have demonstrated that EPD of chitosan nanoparticles form insoluble chitosan films of uniform thickness on substrates of complex shapes [44]. CSnp may also be modified to achieve desired electrochemical and biological properties.

The antibacterial properties of CSnp may be attributed to the electrostatic attraction between the CSnp and the negatively charged bacteria cell membrane. This interaction may alter cell wall permeability resulting in the leakage of the proteinaceous and other intracellular components, which may eventually lead to cell death [37, 38]. It has also been shown that deposition a layer of CSnp film on dentin surfaces may impede bacterial re-colonization [27]. Despite numerous advantages, the effectiveness of CSnp remains limited to the ability to effectively deliver the particles to hard-to-access areas within the tooth such as the root canal system in which the bacterial biofilms exist.

Effective inhibition of biofilm bacteria by CSnp generally requires a high concentration of the nanoparticles to remain in contact with the bacteria for a long duration [27, 32]. Previous studies by the inventors have demonstrated that the rate of bacterial killing is depended on concentration and time. Chitosan nanoparticles (10 mg/ml) have shown complete killing of bacteria after 8 hours (27). If attempting to treat extraradicular biofilms in this manner, obtaining high concentrations of CSnp periapically is paramount. Conventional endodontic methods are not able to supply a concentration of CSnp that is high enough to inhibit development of bacterial biofilms as evidenced by the following example. CSnp may be introduced to the tooth using mechanical irrigation techniques used for apical extrusion of debris. Typically, the apical fluid pressure applied must exceed the venous pressure of the facial veins [39]. A needle delivering apical fluid may be intentionally made to engage the dentin and increase the apical pressure to deliver a suspension of CSnp. The particles may then infuse into the surrounding tissues causing CSnp uptake through portals of entrance into the facial venous vasculature. However, the approach of using conventional positive pressure irrigation may not provide a concentration of CSnp periapically to address the development of bacterial biofilms. Furthermore, this increased pressure approach may also lead to tissue damage and post-operative pain.

As described earlier, particles in suspension may move at characteristic velocities, determined by their charges, size and mobility under the influence of a non-uniform electrostatic field. As such, rather than applying positive pressure needle irrigation techniques, the inventors have discovered that electrophoretic deposition (EPD) of charged particles may be used to take advantage of the interaction between the charged particles and an applied electric field. Electrophoresis may transport charged substances, such as nanoparticles in a suspension, onto substrates with complex shapes and surface areas that conventional irrigation techniques cannot. The first model of EPD kinetics was proposed by Hamaker in 1940 [47] for electrophoretic cells with a planar geometry. It relates the deposited mass (M) with slurry properties, such as suspension concentration (Cₛ), and electrophoretic mobility (μₑₚ), with the physical conditions imposed on the system such as electric field (E), deposition surface area (S) and deposition time (t). Equation (4) illustrates this relationship:

M=CₛμₑₚSEt (4)

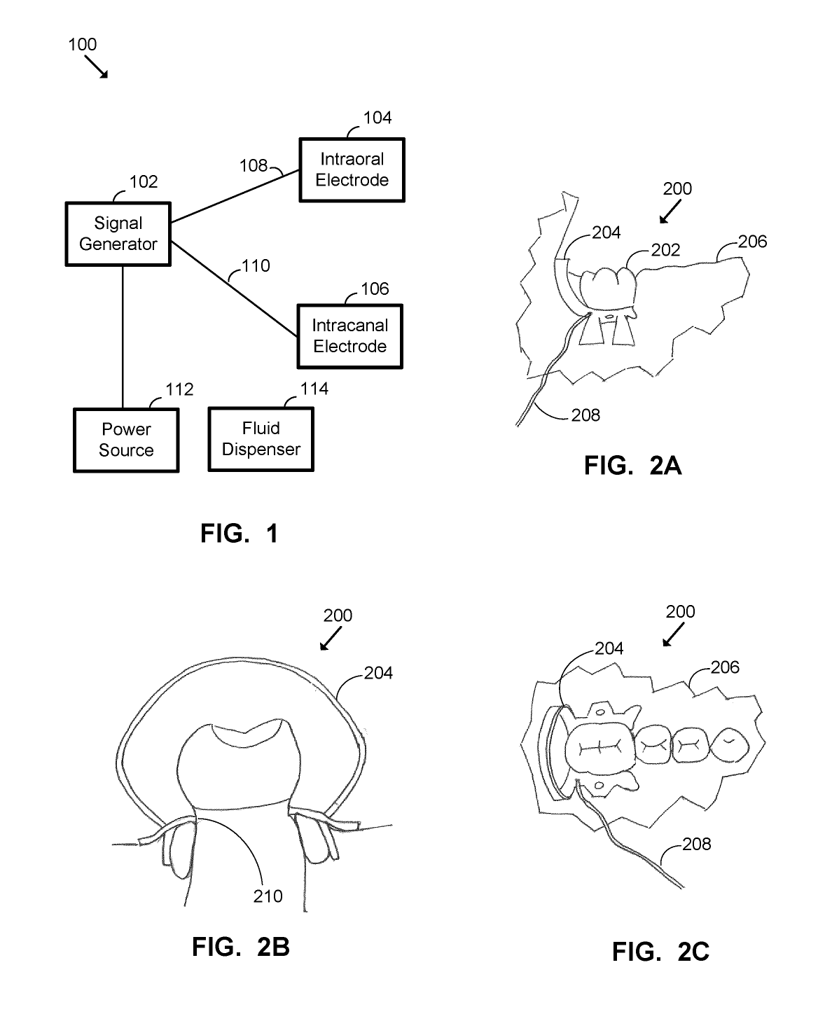

The delivery of particles sensitive to an externally applied electric field based on the above relationship may be realized using the system described in FIG. 1. Referring now to FIG. 1, shown therein is a block diagram of an example embodiment of system 100 for delivering particles to the root canal system of a tooth in accordance with the teachings herein. The system 100 comprises a signal generator 102 and at least one pair of electrodes including an intraoral electrode 104 and an intracanal electrode 106. Wires 108 and 110 are electrical leads that are used to couple the electrodes 104 and 106 to the signal generator 102. The signal generator 102 is coupled to a power source 112. The electrodes 104 and 106 are deployed to the root canal system of a tooth that is receiving treatment. The intraoral electrode 104 is deployed to a region that is adjacent and exterior to the tooth receiving treatment and is in contact with gingival soft tissues, hard tissues, or the periodontal ligament. The intracanal electrode 106 is deployed to the root canal of the tooth receiving treatment. The system 100 also comprises a fluid dispenser 114 which dispenses a solution of particles to the root canal of the tooth being treated. As described in further detail subsequently, in some embodiments, application of an electric field alone may be implemented for the treatment of bacterial infections. In other embodiments a combination of electric field and antimicrobial particles may be introduced to the tooth receiving treatment. An electrical signal is then generated by the signal generator 102 and applied to the two electrodes 104 and 106 to set up an electric field therebetween to drive the charged particles in the dispensed solution and deposit the charged particles to desired target areas of the tooth.

The power source 112 may be a regulated power supply configured to generate various frequencies (AC or DC) which results in an electric field between the electrodes 104 and 106. In some embodiments, electrical pulses may be generated having a desired frequency to generate the electric field. It has been identified that pulsed frequencies or applications of block current may enhance bacterial detachments. Specifically currents of 15 μA, 60 μA and 100 μA within a frequency range between 0.1 Hz-2 Hz and duty cycle between 5%-50% may be applied to induce detachment [61]. The shape of the electric field may depend on the electrical characteristics of the surrounding environment (e.g. tooth and various oral tissues). However, in some embodiments, the field may be altered by adjusting the placement of the electrodes and the polarity of the field. The power supply 112 may be configured to maintain a constant voltage or current output notwithstanding changes of electrical characteristics of the load, leads 108 and 110, temperature, etc. to ensure that a continuous driving force is applied to the charged particles. For example, if a constant voltage output is desired, then the output current may be adjusted by the power source 112 when a change in load resistance (i.e. variation in electrode placement) is detected in order to maintain a constant output voltage. Conversely, if a constant current output is desired, then the output voltage may be adjusted by the power source 112 when a change in load resistance is detected to ensure constant current output. In some embodiments, the power source may be a solid state switching power supply. In other embodiments, the power supply may be a bench top power supply. In yet other embodiments, the power supply comprises a low voltage battery coupled to a step-up voltage converter.

The intraoral electrode 104 may be placed within the mouth but outside of the tooth. For example, the intraoral electrode 104 may be placed in contact with the oral soft tissues surrounding the tooth receiving treatment. The intraoral electrode 104 may be applied using a modified dental rubber dam clamp or a ring or band that is placed around the crown of the tooth being treated, extending into the gingival sulcus. This configuration allows the intraoral electrode 104 to be directly electrically coupled with the periodontal ligament space.

The intracanal electrode 106 is inserted within the root canal system of the tooth being treated. The intracanal electrode 106 may be designed to output the desired electric field, and minimize aberrant currents. The resultant low current electric field created between the intraoral electrode 104 and the intracanal electrode 106 may drive the charged particle movement with subsequent deposition when resistance is encountered. In some embodiments, the intracanal electrode may be flexible and its dimensions and flexibility may accommodate its passage to the apex of minimally instrumented root canals of various lengths. For example, an electrode between 20-30 mm in length with a tip size between 0.10-0.25 mm and a taper between 0.00-0.04 may achieve this. The electrodes can be made from a metal or metallic alloy comprising of conductive materials such as copper, silver, nickel, titanium, brass or steel. However, sustaining an electric field in an electrolyte requires Faradaic reactions to occur at the anode and cathode. The reactions in question within the tooth are typically electrolysis of water, which generates hydrogen peroxide, hydrogen ions (acid) and hydroxide (base) as well as oxygen and hydrogen gas bubbles. The generated hydrogen peroxide and/or pH changes can adversely affect biological cells, while gas bubbles can obstruct the microfluidic system. These problems can be alleviated by using alternative electrode materials such as conjugated polymers which can undergo the Faradaic reactions themselves, and thereby dramatically reducing electrolysis. Therefore, as an alternative to metal or metallic alloy electrodes other fouling-resistant or electrolysis reducing materials such as graphite or conductive conjugated polymers may also be used.

Suitable particles can be used for one or more of disinfection, medicinal purposes, anti-inflammatory, analgesic, regenerative, re-mineralization, root filling and biological healing by tissue regeneration/organized repair. Antibacterial nanoparticles may be classified as organic compounds, including but not limited to, Poly-∈-lysine, Quaternary Ammonium Compounds, Cationic Quaternary Polyelectrolytes, N-Halamine Compounds, Polysiloxanes, Benzoic Acid, Phenol, and p-Hydroxy Benzoate Esters, Quaternary Phosphonium or Sulfonium Groups, Triclosan, 5-Chloro-8-hydroxy-quinoline, Peptides, Organometallic Polymers, Polymeric Nanosized Antimicrobials and Chitosan) or inorganic materials (including metals and metal oxides such as silver (Ag), gold (Au), iron oxide (Fe₃O₄), titanium oxide (TiO₂), copper oxide (CuO), zinc oxide (ZnO), magnesium oxide (MgO), nitric oxide (NO), aluminum oxide (Al₂O₃)) among others.

In some embodiments, the charged particles may be suspended in a carrier material or a “vehicle” in the fluid dispenser 114 and then delivered to the root canal. It may be generally desirable for the charged particles to be suspended in a carrier having a high dielectric constant (˜50-80), low conductivity (5-500 μS/m) and low viscosity (0.5-5 cP) to facilitate movement and deposition of the charged particles to the target areas. One example of such a suitable carrier liquid is deionized water. It has a high dielectric constant (˜80), a low conductivity (5.5 μS/m) and low viscosity (1 cP) at 20° C. Under circumstances where the carrier has a low a dielectric constant, deposition may fail due to insufficient dissociative power. On the other hand, with a carrier having a high dielectric constant, the high ionic concentration in the carrier may reduce the electrophoretic mobility of the charged particles [48]. Low conductivity may be desirable for EPD because it is generally understood that motion of the charged particles may be reduced if the carrier vehicle is too conductive. However, if the overall suspension is too resistive, the particles may build up charge, which reduces the stability of the suspension (i.e. making it difficult to maintain a non-agglomerated and homogenous slurry of charged particles). The stability of the suspension may be governed by the total interparticle potential energy. Dominating forces include van der Wall attractive forces, double layer (electrostatic) forces and steric (polymeric) forces. In order to obtain a well stabilized suspension, the particles in the suspension preferably exhibit sufficient repulsive forces to offset the van der Waals attraction. A large zeta potential of the particles in suspension plays a key role in stabilization of the suspension due to increased repulsive forces between particles.

In general, the deposition time may be decreased by increasing the magnitude of the applied fields. However, the quality of the resulting deposit may suffer. For example, less intense fields (25-100 V/cm) may generate more uniform coatings while higher strength fields (>100 V/cm) may cause the film quality to deteriorate [49]. Furthermore, a higher applied field may cause turbulence in the suspension, disturbing the deposition process. In addition, particles may be moving so fast that they may not find enough time to settle in their optimal positions to form a close-packed structure [48].

The role of time on EPD kinetics may be a linear relationship between time and deposition mass during the initial EPD period. However, this high deposition rate may subsequently decrease and reach a plateau after very long deposition times (<10 min) [49-51]. This decrease may be due to the accumulation and build-up particle deposits on the surface of the electrodes 104 and 106. The insulating nature of this deposit may decrease the electric field strength driving EPD process despite the voltage remaining constant at the power source 112 [52].

Referring now to FIGS. 2A-2C, shown therein are side, cross-sectional and top views, respectively, of schematic representations of an example embodiment of an intraoral electrode arrangement 200 in accordance with the teachings herein. It should be understood that there may be other electrode arrangement designs that may be used with the intraoral electrode. The electrode arrangement 200 comprises a rubber dam clamp 204 and a dental rubber dam 206. An electrode lead 208 may be attached to the dental clamp 204, which is put in place to achieve two objectives. The first objective of the dental clamp 204 is to retain the rubber dam 206 in place as shown in FIGS. 2A-2C. The second objective of the dental clamp 204 is to deliver current from the electrode lead 208 into the area of the sulcus or to the gingiva and/or mucosal soft tissues 210 that surround the tooth 202 designated for treatment. The dental clamp 204, which in essence is the intraoral electrode, may come in direct contact with the gingival tissues or penetrate the oral tissues surrounding the tooth 202 requiring treatment. The rubber dam 206 may provide the necessary aseptic conditions needed during root canal therapy and may also provide an insulating barrier to prevent aberrant currents and short circuits in the overall system 100.

In at least some embodiments, an adhesive conductive hydrogel (not shown) may also be used to maximize contact area and decrease current density at the epithelial contact point.

In at least one alternative embodiment, the intraoral electrode arrangement 200 may be modified to include a needle that is coupled to the dental clamp 204 and penetrates into the bone in the periapical area of the tooth 202.

In at least one other alternative embodiment, the intraoral electrode arrangement may be a modified root canal file which is placed within the root canal and in close proximity to the apical foramen of the tooth 202.

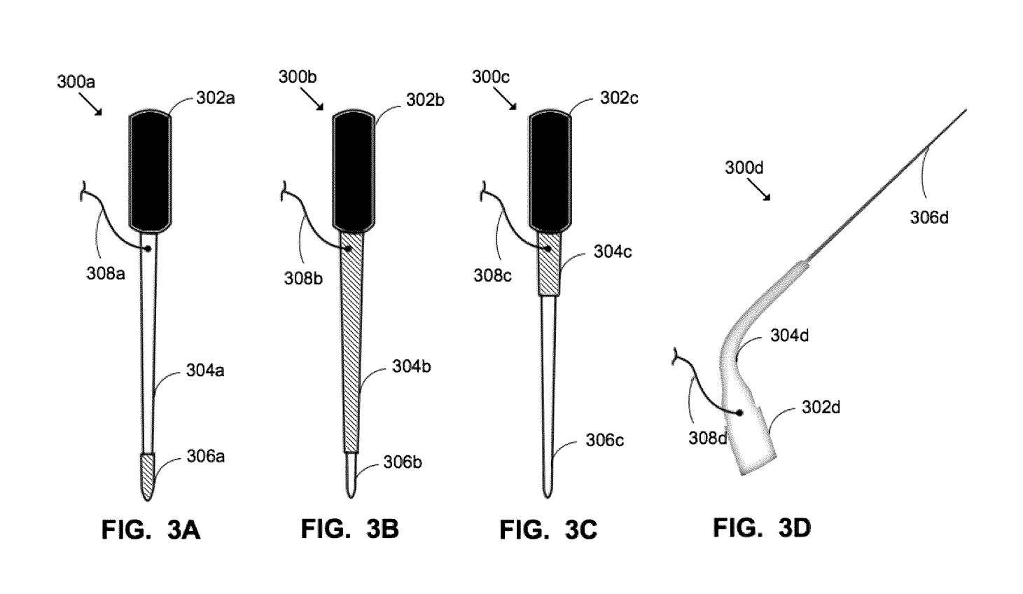

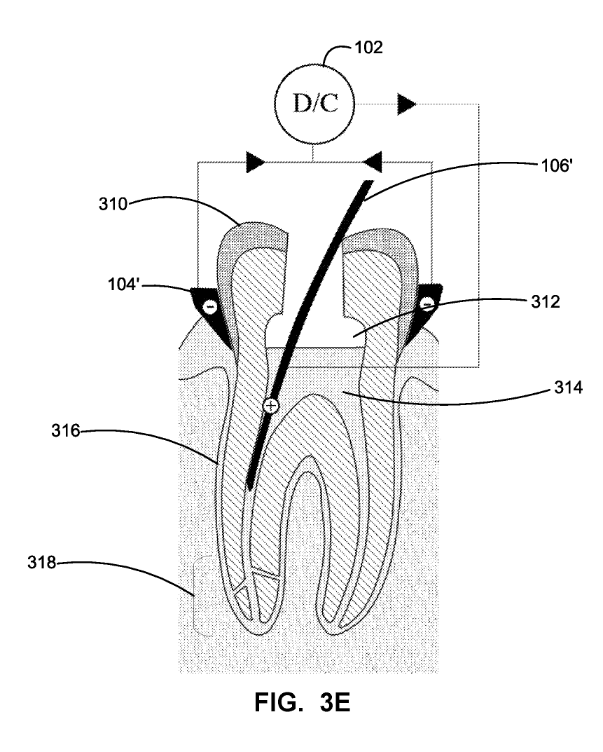

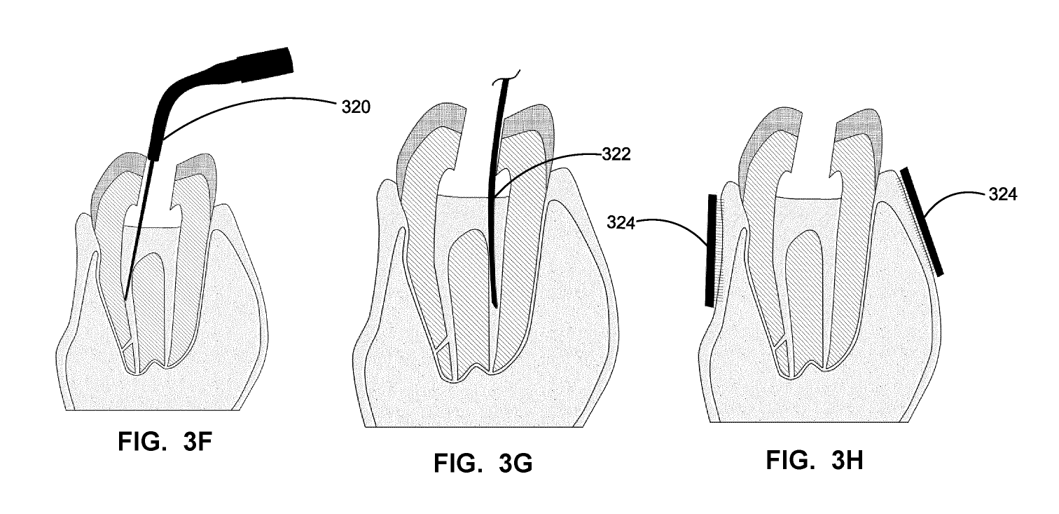

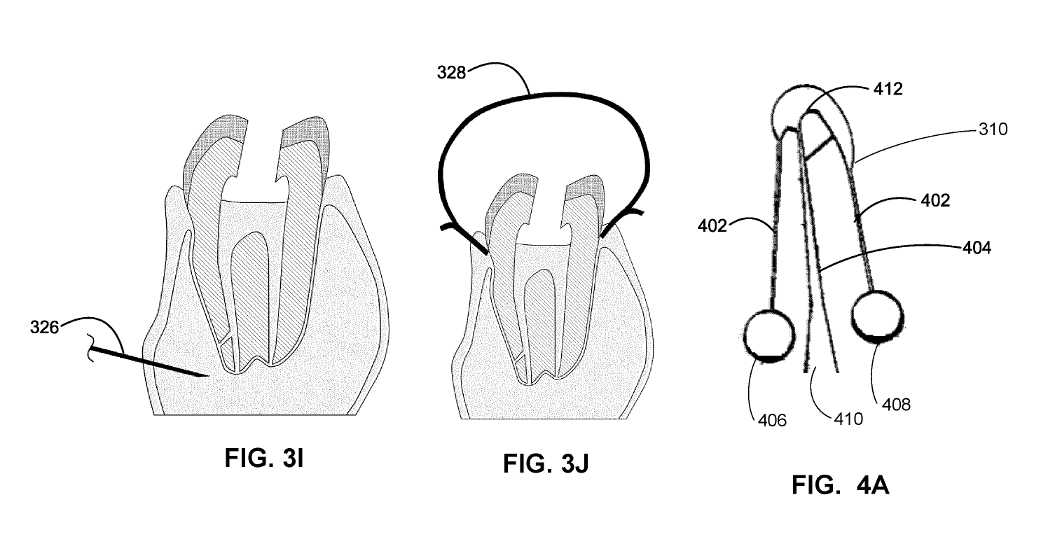

Referring now to FIG. 3A-3D, shown therein are alternative example embodiments of an intracanal electrode. FIGS. 3A-3C show hashed and hash-free regions. Hashed regions denote areas of the electrode covered by an insulating material while hash-free areas denote exposed conducting surfaces. It should be understood that there are a variety of different variations or configurations that may be possible for the intracanal electrode. The intracanal electrode is used to deliver electric current within the root canal space and drive the charged particles or any other substance from the root canal space to an external root surface or the periapical region.

Referring now to FIG. 3A, an example embodiment of an intracanal electrode 300a may be a modified conducting endodontic file covered with an insulating sheath 306a, except on the conductive shank region 304a where the electric current/field for EPD may be generated. The intracanal electrode 300a may comprise an insulating handle 302a with an aperture for connecting a conductive wire 308 to an inner conductive conduit which is covered by the insulating sheath 306a near the tip of the instrument. The insulating sheath 306a may be made of any suitable non-conducting material such as rubber, for example. The functional portion of the instrument 304a/306a may vary in length, diameter, taper and cross-section to accommodate different sizes, shapes and lengths of root canals, since some root canals may be larger or longer than others. In some embodiments, the intracanal electrode 304a/306a may also be connectable to a conventional dental ultrasonic unit. The ultrasonic unit may operate to produce an ultrasonic wave (>25 kHz) or a sonic agitation wave (1 kHz-10 kHz). Various portions of the conductive wire 308 may also be insulated to prevent external interference which may affect the desired electric field. This embodiment may be utilized to generate an electric field laterally driving particles towards the dentin and achieve EPD on the dentinal walls.

Referring now to FIG. 3B, shown therein is an alternative embodiment of the intracanal electrode 300b that also comprises an insulating handle 302a, an insulating sheath 304b, a conductive tip/end region 306b, and a wire 308b. In this case, the same insulating material may be used for both the handle 302b and the insulating sheath 304b. This embodiment may be utilized to generate electric field lines through the apical foramen or other ramifications such as lateral or accessory canals and drive particles into the periapical area.

Referring now to FIG. 3C, shown therein is another alternative embodiment of the intracanal electrode 300c that also comprises an insulating handle 302c, an insulating sheath 304c, a conductive tip/end region 306c, and a wire 308c. In this case, the sheath 304c is much shorter than the conducting tip portion 306c. This embodiment may be useful in achieving similar electric fields as in FIGS. 3A and 3B while preventing aberrant currents (short circuits) in the cervical portions of the teeth.

Referring now to FIG. 3D, shown therein is another alternative embodiment of the intracanal electrode 300d that may be a modified conductive ultrasonic tip consisting of a base 302d that can be attached to a piezoelectric dental ultrasonic unit, a curved shank 304d that may be insulated, a portion 306d that is inserted within the root canal and a wire 308d. Any portion of this electrode 300d may be covered by an insulating sheath to aid in electric field generation. This embodiment may be useful in delivering ultrasonic energy before, during or after application of the electric field.

An alternative embodiment of the intracanal electrode may consist of a conductive hollow needle of various gauges and lengths connected to an insulated lead or wire and a fluid reservoir. The needle may be open-ended, side-beveled, or side-vented and portions of the needle may be insulated to achieve electrical fields directed towards the lateral or apical aspects of the root canal. This embodiment may be useful in delivering and replenishing the desired particle suspension into the root canal and simultaneously or subsequently delivering electric current within the root canal space.

In general, in these various example embodiments, the intracanal electrode may be held at a static position or agitated mechanically or using sonic or ultrasonic agitation, for example as shown in FIGS. 3A and 3D, respectively.

Referring now to FIG. 3E, shown therein is a diagram illustrating the deployment of an intraoral electrode 104′ and an intracanal electrode 106′ in an example embodiment. The intracanal electrode may be deployed to various depths within the root canal space and may even be placed deep into the canal in such a way that the tip of the instrument extends beyond the apical foramen. The intraoral electrode 104′ may be deployed as previously described and come in contact with the tooth on the surface of the enamel 310. Furthermore, the intraoral electrode 104′ may be assigned to be the electrical return path (i.e. negative terminal) back to the power supply 112. To prepare the tooth for insertion of the intracanal electrode 104′, the root canal may be prepared using standard techniques to create an opening large enough to permit a solution, having the desired charged particles in suspension, to be delivered into the root canal system (e.g. the primary canal to be treated and possibly any adjacent accessory canals, isthmuses or ramifications) and for the deployment of the intracanal electrode 106′. It should be noted that only a minimally enlarged opening 312 may be required for the root canal for delivery of the charged particles 314 and the intracanal electrode 104′. Therefore, the opening 312 may generally be smaller than a conventional root canal opening because of the effectiveness of the method to combat bacteria in the root canal system as described in accordance with the teachings herein.

The use of a minimally enlarged opening 312 is consistent with the philosophy behind minimally invasive endodontics (MIE) which recognizes the value of maintaining the greatest amount of the original tooth structure while achieving the desired treatment goals. Endodontic treatments that maximize the preservation of the original tooth structure may improve the fracture resistance of teeth after they are treated. For example, a tooth fracture following root canal treatment may lead to tooth extraction and is a significant burden in the field of endodontics. While adhering to the concepts of MIE promotes preservation of relevant dental structures, practically speaking, doing so may impede a clinician's ability to effectively deliver chemical irrigants within the root canal, thereby impeding adequate root canal disinfection, using conventional treatment techniques. As a result, conventional endodontic preparations may often require significant removal of the original tooth structure, around the pulp chamber, around canal orifices, and within the root canals. Removal of this sound tooth structure may undermine the fracture resistance of the tooth under functional loads. Advantageously, the teachings described herein utilize electrokinetic particle transport to apical regions through small access cavity preparations and through minimally enlarged canals satisfying the principle of MIE. The approaches described in accordance with the teachings herein may also allow for preservation of sound tooth structure during root canal treatment.

The intracanal electrode 104′ may be inserted into the root canal to the desired depth after introduction of a solution of the charged particles. In some embodiments, the charged particles may be delivered within the root canal space in such a way that the particles are extruded from the root canal space to come into contact with the periodontal ligament space 316 (described in more detail below). This extrusion of particles may be achieved by placing a delivery needle in close proximity, or beyond the apical foramen when introducing the solution. In doing so, a continuum of conductive material (e.g. between the medium in which the charged particles are suspended and the periapical tissue fluids) may be established to electrically link the intracanal electrode 104′ to the intraoral electrode 106′. An electric field is created between the intraoral electrode 104′ and the intracanal electrode 106′ with sufficient strength to provide sufficient driving forces to deliver the charged particles to various locations within the tooth and just outside of the tooth. The structure and arrangement of the tooth's hard tissue and insulating structures of the jaw may further serve to guide electrokinetic flow of the charged particles toward the apex of the tooth through the apical foramen or any patent apical portals and into the periodontal tissues adjacent to the root tip.

As the charged particles are driven from one electrode toward the other electrode, the charged particles may be drawn toward the apex 318 of the tooth which increases the concentration of antimicrobial particles in areas of the tooth that are generally difficult to access using conventional techniques. Driving the charged particles toward the apex of the tooth may provide a number of benefits. For example, antimicrobial particles may be delivered to the apical delta, which consists of small, branched accessory canals in the apical portion of the root canal. Within an infected tooth, the apical delta region is known to harbor bacteria and it is generally difficult to clean using conventional methods. It is also generally believed that a major cause of root canal failure is the persistence of microorganisms in the apical portions of root filled teeth. Whenever endodontic treatment is not successful, this area is often surgically removed, to eliminate the apical delta, and maximize the chance of successful healing. Due to the small and complex anatomy in this region, the apical delta it is often impossible to fully clean using conventional methods.

The ability of the system and methods described herein to target bacteria in the apical delta and on the extraradicular root surface is of great clinical benefit since the success of endodontic treatment may be increased and the need for apical surgery procedures may be reduced. Using the teachings herein, as the antimicrobial particles are drawn apically through channels of increasingly narrower diameters, their local concentrations would increase, thus increasing their efficacy. Furthermore, as particle concentrations increase, they may begin to form clusters (particle trapping) and blocking of these regions may occur. This space-occupying feature may prevent future bacterial growth in these regions.

Another benefit of the deposition of charged particles with antimicrobial properties according to the teachings herein is that the deposition of charged particles may be used to treat extraradicular bacterial biofilm. For instance, bacteria of endodontic origin may also be found outside of the root canal where they can develop a biofilm-like structures on the cementum surface of the root (e.g. adjacent to the apical foramen) [52], or as cohesive colonies within a periapical lesion [10, 11]. A number of studies have indicated that failed endodontic treatment may be caused by bacterial biofilms that are present on the external root surface and are capable of evading host defense mechanisms. These biofilms often cannot be addressed by conventional endodontic procedures and may continue to exhibit their pathogenic effects after the treatment has completed [12-14]. Moreover, recent studies utilizing advanced molecular techniques together with direct visualization by scanning electron microscopy (SEM) imaging and light microscopy provide evidence that such extraradicular biofilms are not uncommon, especially in cases that do not respond to conventional endodontic treatment [15-17]. The ability to deliver antimicrobial particles to target bacteria in the apical delta and on the extraradicular root surface, in accordance with the teachings herein, may therefore increase the success of endodontic treatment as well as eliminate the need for some types of apical surgery procedures.

Referring now to FIG. 3F, shown therein is a diagram illustrating example embodiments in which different styles of intraoral and intracanal electrodes may be deployed, in accordance with the teachings herein. For example, in some embodiments, an ultrasonic intracanal electrode 320 may be used. Such an electrode may be produced by equipping a standard intracanal electrode 104 with an ultrasonic tip capable of simultaneously transferring ultrasonic and electrical energy into the root canal containing charged particles. For example, a customized electrified ultrasonic tip (e.g. those used in endodontics to deliver ultrasonic energy to the root canal) may be fitted onto a conventional piezoelectric unit commonly used in dentistry. Such a tip may be conductive and may be manufactured so that it is connected to an electrical lead to operate as an intracanal electrode. In an alternative embodiment, a clasp, consisting of a conductive wire bent to form a hook and encased in a plastic shell, may be attached to existing ultrasonic tips to deliver electrical energy and achieve similar effects.

In other embodiments, a needle-type intracanal electrode 322 may be used, as shown in FIG. 3G, which may include a hollow tube or needle capable of delivering the desired particles apically. Alternatively, the needle-type intracanal electrode 322 may be fabricated using a modified endodontic file. As discussed previously, a hollow-type electrode may be used to deliver particles to the canal system or replenish particles as necessary during treatment.

In other embodiments, a microneedle intraoral electrode 324 may be deployed on the gingiva as shown in FIG. 3H. A conductive plate consisting of an array of microprojections may be placed onto the gingiva and used to penetrate the skin barrier and deliver current into the subcutaneous tissues. This embodiment may be useful in overcoming the insulating properties of the skin.

In other embodiments, an intraosseous intraoral electrode 326 may be used which is deployed into the bone structure near the tooth as is shown in FIG. 3I. This electrode may penetrate through the gingiva, cortical bone and enter into the cancellous bone apical to the root of the tooth to be treated. This embodiment may be useful in overcoming the insulating properties of the skin and generating electrical field lines directed towards the apical portion of the tooth.

In other embodiments, a modified rubber dam clamp can serve as the intraoral electrode as is shown in FIG. 3J. This electrode may come into contact or penetrate gingival tissues on the buccal (cheek side) and lingual (tongue side) region of the tooth designated for treatment. This embodiment may be useful for delivering current from the intracanal electrode into an area of the sulcus or to the gingiva and/or mucosal soft tissues that surround the tooth designated for treatment.

### Computational Modelling

The effectiveness of delivering charged particles via EPD according to the teachings herein may be predicted using computer-based simulation by modelling the electric field and the movement of the charged particles. Numerical modeling techniques known to those skilled in the art may be used. For example, the interaction between an applied electric field and charged particles may be modelled using the COMSOL multiphysics software package (Version Palo Alto, Calif.). In other instances, the same analysis may be done using custom software and Monte Carlo techniques.

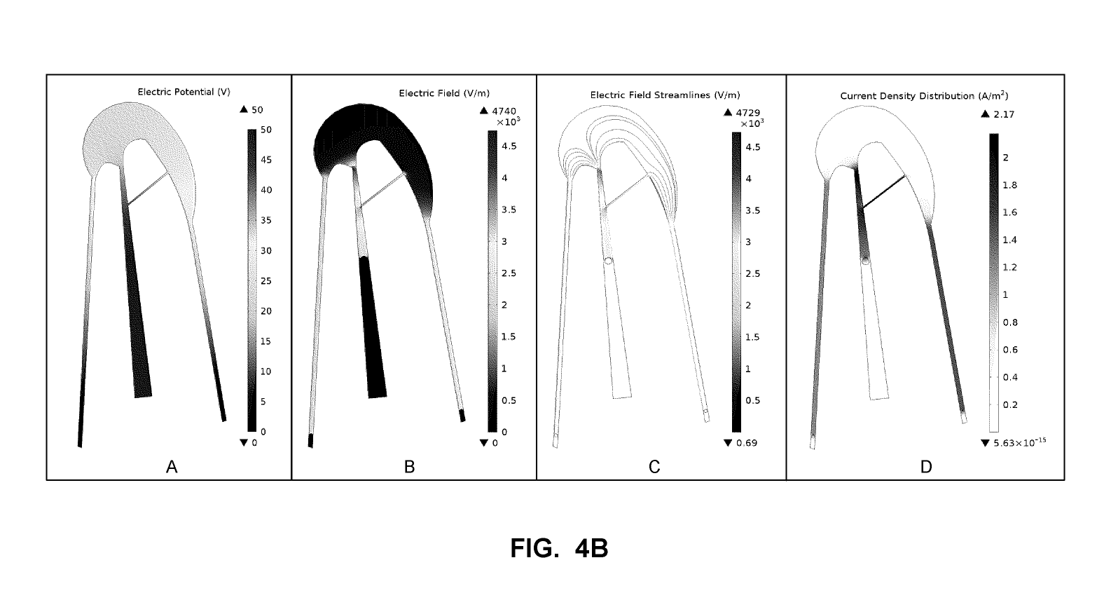

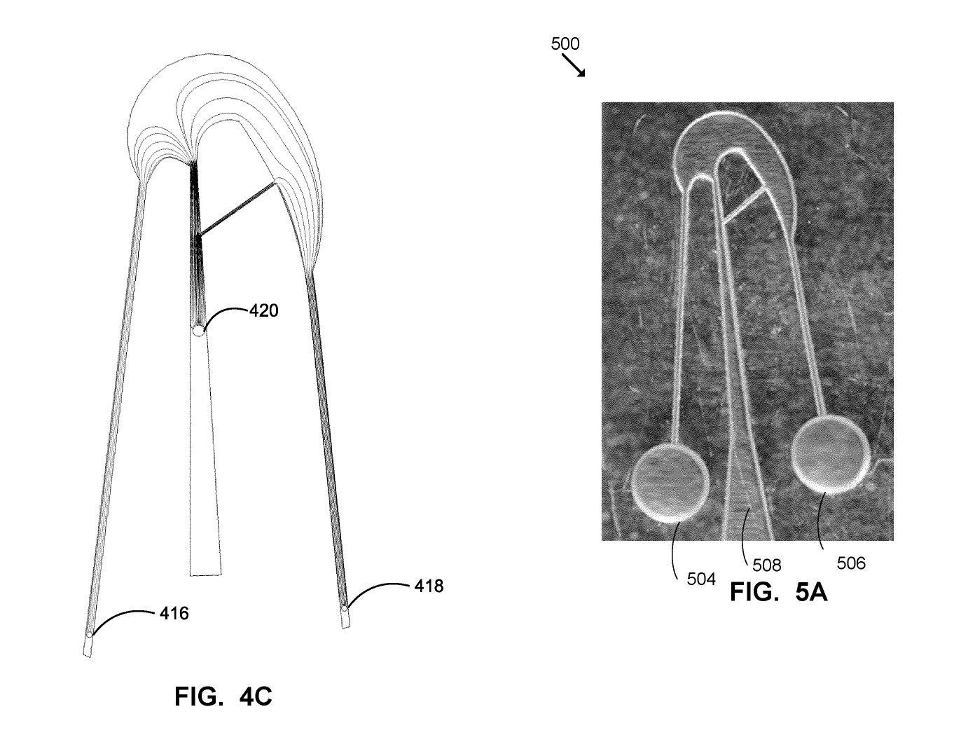

Referring now to FIGS. 4A-4C, shown therein is an example of an EPD simulation model to predict and visualize the electric potential, electric field streamlines, electric field distribution, and current density distribution in a two-dimensional structure representative of a coronal cross-section of a maxillary central incisor and periodontal ligaments 402. FIG. 4A shows an outline diagram of the simulation geometry, based on an extracted central incisor, with a lateral canal in the apical ⅓ and an area of periapical bone loss 310. For the simulation parameters, the width of the periodontal ligament (PDL) space 402 may be set to 0.2 mm [62], and the length of the canal 404 may be set to 13 mm. The width of the root canal space 404 may be sufficiently large to accommodate a size 25, 0.06-tapered root canal file. The size of the periapical lesion may be selected arbitrarily, but designed with an epicenter encompassing the apical foramen and the lateral canal.

Fluid reservoirs 406 and 408 may be used to store the particles to be delivered by EPD. Boundaries of the simulation geometry may be defined as insulators and the electrodes may be defined to be made from conductive material such as stainless steel (UNS s17600). The luminal contents may be defined as water at 20° C. with a known relative electrical permittivity ∈=80 [63]. An extra fine mesh may be used for numerical modeling. The model may be assumed to be a closed microfluidic system and the electric potential may be assumed to be 50V DC.

In actual practice, it may be possible to apply a potential difference of about 20 to 70V DC or AC, although a potential difference of about 50V may be preferable. More or less current may be used depending on one or more of the charge of the charged particles used (e.g. zeta potential), size of the charged particles used, or the conductivity parameters of the suspension solution (i.e. carrier solution).

In addition, the potential difference may be applied for about 1 to 15 minutes and in some cases it may be about 10 minutes. The amount of time that the potential difference is applied depends on one or more of the charge of the charged particles, the size of the charged particles and certain parameters of the suspension solution. These parameters will dictate how fast the particles will move which can affect the amount of time that the potential difference should be applied for.

Shown in FIG. 4C are the placement of three electrical contact points 416, 418, 420 (circles). Two are located in the cervical portion of the PDL space 416, 418 (cathodes), and a third contact point is located in the middle portion 420 of the root canal (anode). These circles represent possible arrangements of the intracanal and intraoral electrodes in a clinical scenario. The distribution of the electrical potential across the three electrodes may be visualized in panel (A) of FIG. 4B. Simulations of the computational model described herein shows that upon application of an electric potential between the two electrodes, a non-uniform electric field distribution is produced. For example, the electric field lines in panel (C) of FIG. 4B pass through the apical constriction and the lateral canal running perpendicular to the simulated root canal walls, and thus may be perpendicular to most biofilm surfaces. A more detailed view of the distribution of electric field lines is shown in FIG. 4C. The arrangement of hard tissue and insulating structures of the jaw may be viewed as obstacles in the path of the electric field, thereby creating a non-uniform field distribution. As a result, the electric field lines, as shown in panel (C) of FIG. 4B and in FIG. 4C may be concentrated or “focused” at various locations corresponding to areas of higher non-uniformity. Generally, when viewed from the coronal aspect of the tooth towards the apex, the electric field may gradually converge resulting in intensified electrokinetic flow as the root canal narrows. For example, areas of higher magnitude or “focused” electric fields may develop in regions with narrow cross-sections such as the apical foramen, the lateral canal and the periodontal ligament space, while other “unfocused” areas develop where the insulating surfaces are further apart such as the large area representing apical bone loss (see e.g. reference 412 of FIG. 4A).

For the simulation parameters given previously, the application of 50V DC between the electrodes, as shown in panels (B) and (D) of FIG. 4B, may produce a current of 1.7 mA and an electric field (E) from 5 to 50 V/cm and a current density (J) of 0.05-0.22 mA/cm². Panel (C) of FIG. 4B shows a plot of the electric field lines superimposed on a distribution of the electric field to indicate possible particle trajectory. Therefore the electric field distribution of the present embodiment illustrated in panel (C) of FIG. 4B may be used to indicate regions where charged particles may likely become concentrated or trapped. Panel (C) of FIG. 4B shows that with the present simulated configuration of electrodes, areas of high concentration of charged particles may include the apical foramen, lateral canals and the periodontal ligament space. The charged particles may be “pumped” outside of the tooth through exit portals of the tooth such as the apical foramen and lateral canals. In the case of using CSnp as the charged particles, the modeling shows that field strengths may range from 15-45 V/cm for distributing the charge particles via EPD.

It is generally understood that any sensation or discomfort caused by the application of electrical current to the skin may be caused by the direct electrical excitation of the nerves at the skin [74]. The application of a DC or AC potential difference may cause net ion migration, with depletion or accumulation of ions near the respective electrodes. The altered chemical composition resulting from accumulation or depletion of ions may also generate a sensation that can start under one of the electrodes (anode or cathode). It has been reported that sensory thresholds for DC voltages applied on the hand is around 5 mA, and 45 μA if applied on the tongue [73]. Sensory thresholds for AC voltages depend on the frequency. Pain thresholds may be about 10-20 times higher [70]. Therefore, the applied voltage required to achieve EPD may be chosen so as to operate below levels of current flow known to cause discomfort in a person's mouth. Accordingly, an application of 1.7 mA will be well below the threshold for discomfort.

### Physical Modelling

A physical model comprising a microfluidic device may be used to validate the computational modelling of the transport of charged particles via EPD. The two-dimensional model, as shown in FIG. 4A, in the x and y planes used in the computer simulation may be extended to a three-dimensional microfluidic device having a z-plane. The microfluidic device may be fabricated using polydimethylsiloxane (PDMS) and conventional microfabrication techniques based on soft-lithography processes to study the anatomical relationship of the insulating and conductive structures, as they are situated in the periodontium. The model produces an array of insulating conduits that may direct and focus the applied electric field. The surface charge and insulating properties of PDMS may be suitable for electrokinetic experiments [68], and its transparency in the visible wavelengths may be ideal for direct visual inspection of trajectories and deposition kinetics of charged particles such as CSnp, particularly if the particles are tagged or labeled with fluorescent compounds such as fluorescin isothiocyanate (FITC) [65].

FITC-labeled CSnp (FITC-CSnp) may be synthesized using various methods. One such example method is described herein. Chitosan (1 g) may be dissolved in 100 mL of 0.1 M acetic acid. Dehydrated methanol (100 mL) may then be added and stirred. About 50 mL FITC (2.0 mg/mL) in methanol may be slowly added to the chitosan solution. After a reaction in the dark at ambient temperature for four hours, the FITC-labeled chitosan (FITC-CS) may be precipitated in 0.2 M NaOH to raise the pH to between about 8 or 9. The precipitate may then be centrifuged (40,000 g, 10 min) and washed by a solution comprising acetone:water (3:1 v/v) repeatedly until no fluorescence is detected in the supernatant (Perkin Elmer Inc., luminescence spectrometer LS50B, kexc 485 nm, kemi 520 nm). The FITC-CS compound may then be dissolved in 0.1 M acetic acid and dialyzed in water for 3 days in the dark and freeze dried (Vitris Co. Inc., Gardiner, N.Y.). FITC-CS may be converted to nanoparticulate form (FITC-CSnp) by ionic gelation with negatively charged tripolyphosphate (TPP) sodium ions (67). The charge of the synthesized FITC-CSnp may be evaluated using a Zetasizer (Malvern Instruments Ltd, Malvern, UK).

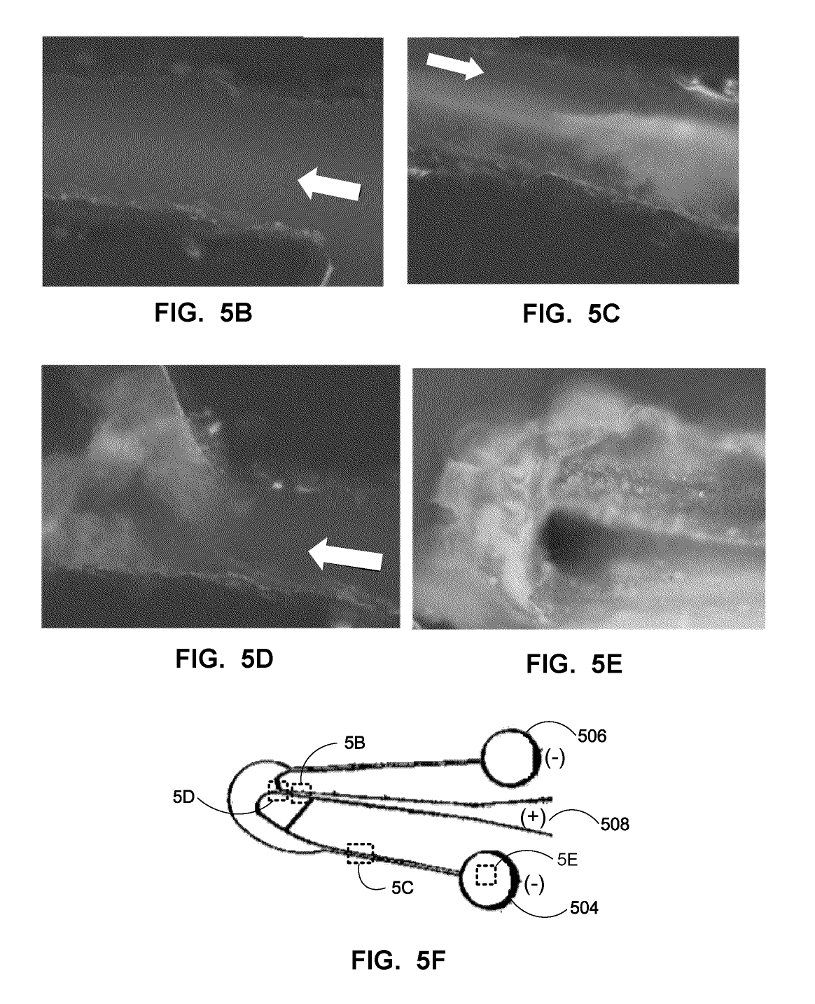

FIGS. 5A-5F illustrate the EPD of particles in the microfluidic device. FIG. 5F shows an outline of the model according to FIG. 4A indicating that in the present embodiment, the reservoirs 504 and 506 are the negative terminals while the root canal space 508 is set as the positive terminal. In some embodiments, needle electrodes may be used such that they may be coupled to the terminals by puncturing the electrode into the PDMS material. Dashed boxes 5B-5E correspond to locations of the microfluidic device from which photomicrographs of FIGS. 5B-5E, respectively, are obtained. FIG. 5A shows a photomicrograph a microfluidic device modeled using the computer simulation geometry of FIG. 4A. Reservoirs 504 and 506 may contain fluorescence-labeled charged particles (here, FITC-labeled CSnp) suspended in an appropriate carrier. Electrode needles may be inserted into the reservoirs 504, and 506 and at various locations within the root canal space 508. Similar to the computer simulation, a potential difference of 50V is applied between the electrodes. The potential difference across the microfluidic device may result in approximately 1-2 mA of current flow. These parameters may be safely measured using standard electronic apex locators (used to determine the position of the apical foramen) and electric pulp testers (used to determine the vitality of a tooth) since these devices may measure potential differences ranging from 0V to 500V, which corresponds to an electrical current ranging from 0 mA to 3 mA [70-72].

FIG. 5B illustrates a laminar flow (indicated by the arrow) of particles in the apical ⅓ of the canal. FIGS. 5C and 5D show that, over time, particles may concentrate and become trapped in the apical foramen (indicated by the arrow) and cause the flow of particles to become chaotic and exhibit characteristics of luminal obstruction. Specifically, FIG. 5C shows that particles may be trapped in a narrow channel such as the PDL and FIG. 5D shows particles becoming trapped at the apical foramen. Areas with diffuse electric fields, such as the coronal ⅓ of the root canal or the periapical lesion, may exhibit slower but streamlined particle flow (not shown). FIG. 5E shows that as particles reach the cathode under the application of a low-intensity current, electrophoretic deposition of antimicrobial particles may be observed.

### Antibacterial Effects of Electrokinetic Delivery and Distribution of Antimicrobial Nanoparticles

Microbiota in primary and persistent endodontic infections may occur predominantly as polymicrobial aggregations, adherent to dentinal surfaces, and embedded in an extracellular polymeric substance (EPS), or biofilms [1-3]. The EPS, with its highly charged and interwoven structure, may deter penetration of antimicrobials by acting as a physical barrier and a chemical neutralizer [4]. Such organization of microorganisms in a diseased environment may provide biofilm bacteria ecological advantages and protection from host responses as well as antimicrobials [5, 6]. Electrokinetic delivery and distribution of antimicrobial nanoparticles, in accordance with the teachings herein, may be able to disrupt the biofilm structure and produce a biological response to the antimicrobials.

The biological effect of planktonic bacteria (i.e. bacteria in a liquid suspension) in response to an applied electric current as observed in a study performed in vitro is presented herein. Overnight cultures of Enterococcus faecalis ATCC 29212 in brain heart infusion (BHI) broth was prepared and centrifuged (3,000 rpm, 10 min), and washed with deionized water. The planktonic bacteria solution was standardized to an optical density of 0.3 at 600 nm. To establish a suitable application of an electric current, two 20 gauge needles serving as electrodes may be fixed approximately 10 mm apart and submerged into 2 ml of bacterial suspension. After 2, 5 and 10 minute treatments with 50V between the electrodes (generating approximately 0.5 mA), a sample (e.g. 100 mL) of the bacterial inoculum was withdrawn and plated onto freshly poured BHI-agar plates after serial dilutions. Bacterial colonies was counted after 24 hours of incubation at 37° C. and expressed as log Colony Forming Units (CFU) per mL.

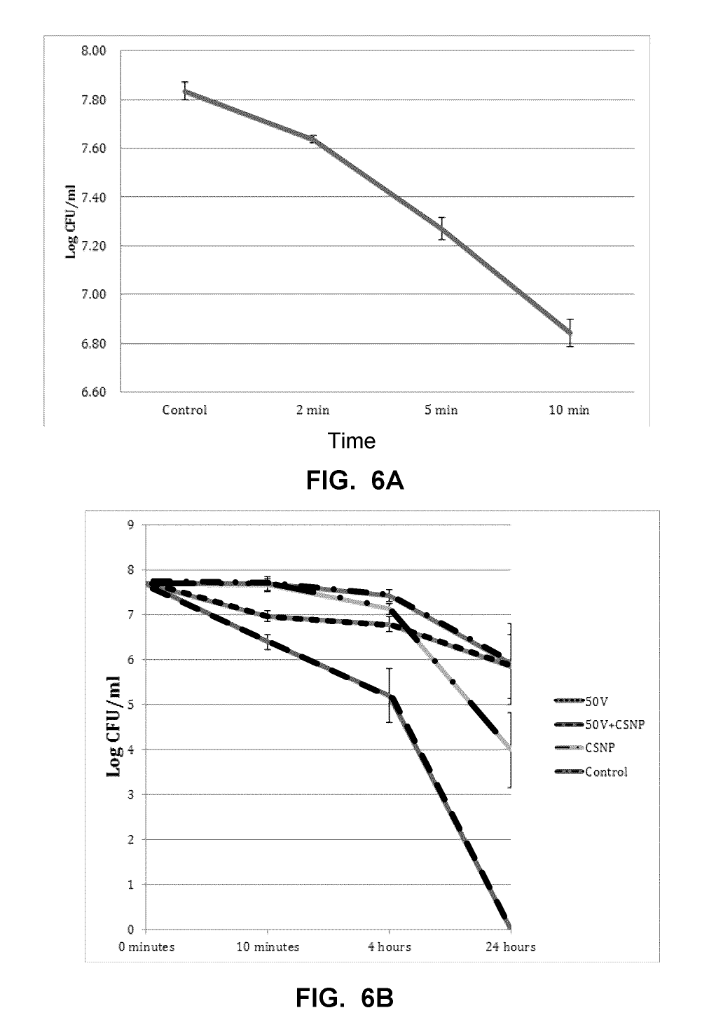

FIG. 6A is a plot which illustrates the in vitro antimicrobial effects of an applied electric current (in the absence of any antimicrobial particles) on colony forming units, as a function of time. The application of electric current alone may reduce the number of viable bacteria as the duration of time increases. A 10-minute application of current, alone, based on the parameters above may produce a one-log reduction in the number of viable planktonic bacteria. It is observed from the plot of FIG. 6A that the number of viable planktonic bacteria, after a treatment with 50V DC (0.5 mA) in the absence of CSnp, may decrease in proportion with the progression of time.

The antimicrobial effects of charged particles such as CSnp combined with electrokinetic delivery may also be studied using planktonic bacterial colonies in vitro. As discussed previously, cationic chitosan nanoparticles may be alternative to conventional endodontic disinfectants and have been shown to be biocompatible and offer broad-spectrum antibacterial properties [27, 32]. Their deposition on dentin surfaces is known to impede bacterial re-colonization [27]. Therefore CSnp may be attractive candidates for electrophoretic transport and subsequent deposition due to their small size, positive charge, and film-forming properties.

To understand the effect of antimicrobial particles combined with an applied electric field, four treatment groups were established. Group 1 was treated with an electric current only (e.g. at 50V DC with a current of 0.5 mA for 10 minutes); group 2 was treated with CSnp at a concentration of 3 mg/ml in conjunction with the application of the same electric field using the same parameters as group 1; group 3 was treated with antimicrobial agents only (CSnp); and group 4 was set as a control group in which no treatment was applied. The experiment was carried out in 24 well plates with electrodes fixed 10 mm apart. The bacterial population was observed over time at 10 minutes, 4 hours and 24 hours.

After 10 min, 4 h and 24 h, a sample (e.g. 100 mL) of the treated bacterial inoculum was observed by plating the sample onto freshly poured BHI-agar plates after serial dilutions and cultured overnight. Bacterial colonies were counted after 24 h of incubation at 37° C. and expressed as log CFU per ml. The observations were carried out multiple times to obtain sufficient data for analysis. The results were subjected to one-way analysis of variance with a post hoc Tukey test. The difference between two test groups may be considered statistically significant if p<0.05.

FIG. 6B is a plot of several curves obtained for the aforementioned test groups which illustrate the antimicrobial effects on planktonic bacteria as a function of time for combinations of CSnp and an applied electric field. While test groups in which CSnp and an electric field were applied, either alone or together, show antimicrobial effects, the combination of electric current and CSnp provides the greatest degree of bacterial reduction. Specifically, group 3 (CSnp only) exhibits a 4-log reduction after 24 hours, while group 2 (CSnp and electric current) suggests near complete bacterial elimination after 24 hours. Thus combining the application of an electric current and antimicrobial particles may produce a larger bacterial reduction than using CSnp-only, using 50V-only or not using either an electric current or antimicrobial particles as seen in the control groups at all observed time points (p<0.01).

The synergistic result of applying both an electric field and introducing antimicrobial particles may be attributed to a combination of bulk removal of bacteria from solution within the initial 10-minute period followed by a subsequently increased efficacy of CSnp towards structurally or metabolically altered bacteria. In this study, both electrodes were removed from the suspension following the application of current thereby removing any viable bacteria electrophoretically attracted and bound to the electrodes from the suspension. It is understood that the concept of attraction and electrophoretic deposition of planktonic bacteria onto electrodes may form a sessile film of microorganisms [75]. The subsequent enhanced killing of bacteria by CSnp may be attributed to altered bacterial homeostasis. It is generally understood that external electric fields can disrupt the electrical equilibria of bacteria thus altering important membrane components [76] and influencing permeability [77], leading to leakage of cellular contents, causing morphological changes [78] and possibly increasing penetration of antibacterial compounds [79].

Antimicrobial effects may also be evaluated using bacterial biofilm structures. Bacterial biofilm development may begin with the physicochemical interaction between microorganisms and a substrate surface. This initial interaction may be attributed to the complex balance between attractive and repulsive forces including Van der Waals forces, acid-base interactions, and electrostatic forces [80], while the integrity of a more mature biofilm may be maintained by the EPS charges.

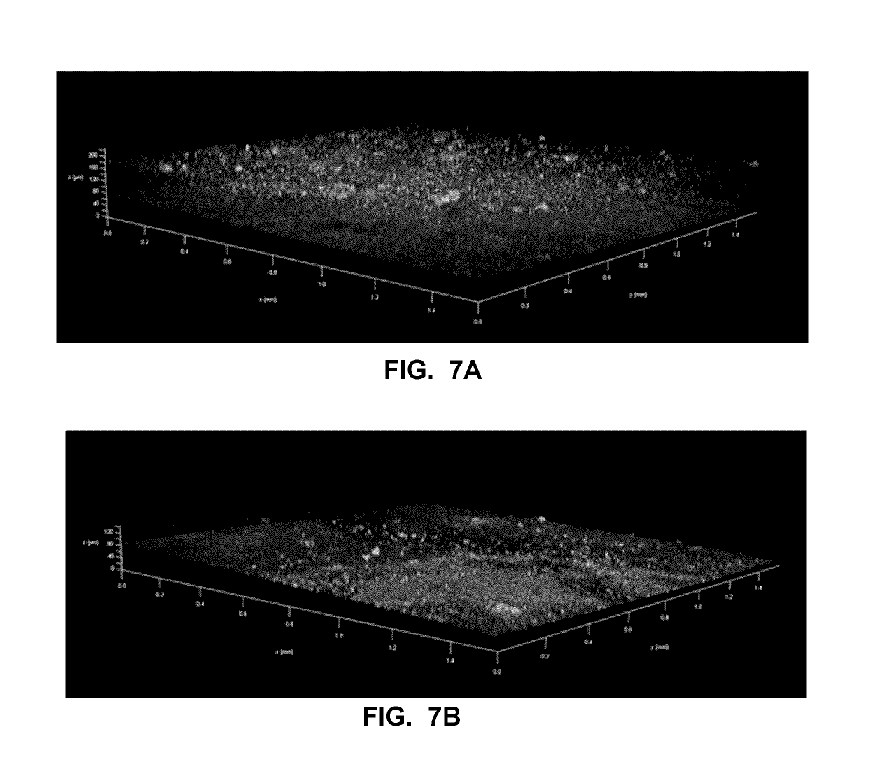

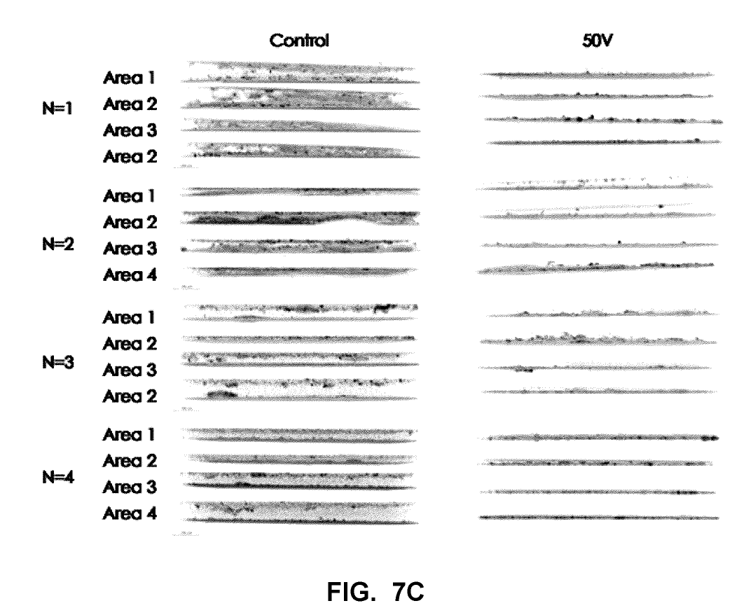

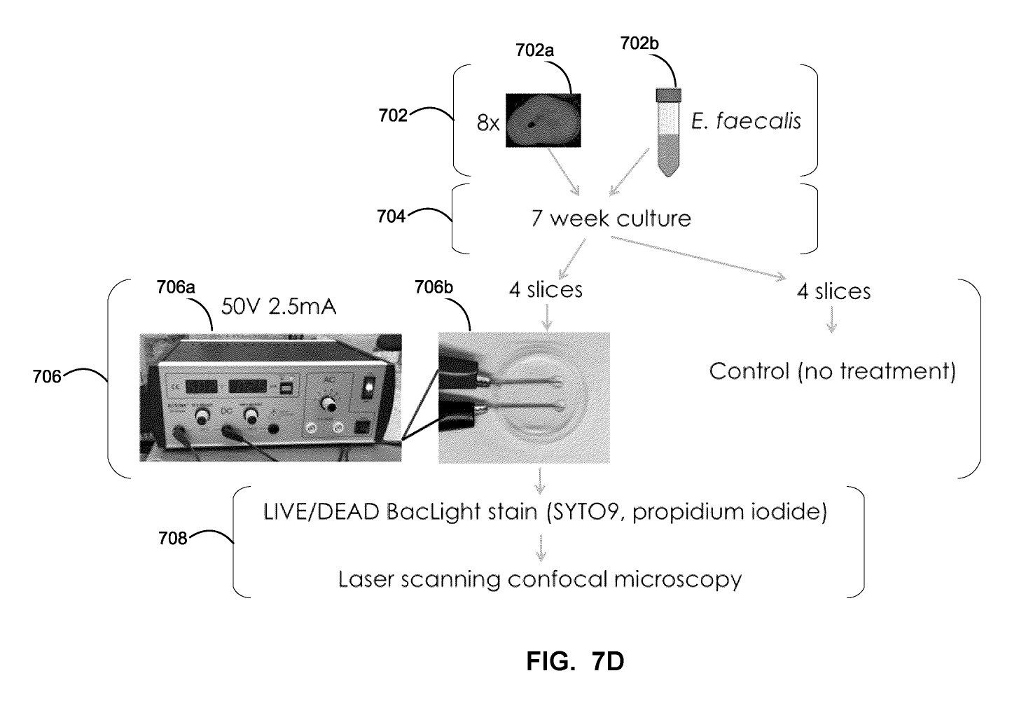

FIGS. 7A-7C show the effects of current on bacterial biofilms. FIG. 7D is a schematic flow diagram illustrating the investigative procedure. The investigative procedure of FIG. 7D is described first, followed by the observations presented in FIGS. 7A-7C. At stage 702, bacterial biofilms were grown over a number of weeks by inoculating dentin slices 702a with E. faecalis 702b. The dentin slices 702a were derived from human maxillary molars and sectioned to generate dentin/enamel slices of 0.5 mm thicknesses. Biofilms were created by adding 1 mL solution of overnight E. faecalis culture in BHI broth to each of eight slices of dentin and incubated in a multi-well plate at 37° C. At step 704 an incubation period of 7 weeks was sufficient to produce a biofilm having a thickness of approximately 93 μm. During the incubation period, fresh medium was replenished every 48 hours to provide a constant supply of nutrients and to remove dead bacterial cells. The biofilm on 4 dentin samples was then treated with the application of an electric field that ran parallel to the bacterial biofilm. The remaining 4 samples may serve as controls. Prior to any treatment, the medium was removed from the well, and the biofilm carefully washed to remove dead cells using sterile deionized water. At step 706 the biofilm samples were divided into similar groupings as described previously. Application of 50V via a power supply 706a to the dentin slices may produce a current flow corresponding to about 2.5 mA. The second treatment group did not receive any treatment and served as controls.

The dentin slices were submerged in a plastic dish 706b containing sterile deionized water and laid horizontally on the surface with the biofilm side facing up between two needle electrodes spaced 10 mm apart. At step 708, after treatment, the dentin slices were stained to identify live or dead cells. For example, 20 μL of a LIVE/DEAD BacLight stain (Molecular Probes, Eugene Oreg.) was introduced to stain the cells in the dark for 15 minutes. The stained cells were then imaged, for example using a fluorescence laser scanning confocal microscope (LSCM; Leica DMIRE2, Germany) to identify cell viability by examining the fluorescence response of the stain. Biofilm structures developed on the dentin slices were imaged at different areas with the LSCM. Images were acquired using a 60× objective lens with oil as the immersion media for visual inspection. An appropriate statistical analysis test such as the student t-test was used to compare the thickness of the biofilm before and after treatment with electric current.

FIGS. 7A and 7B illustrate the fluorescence emission characteristics of an untreated biofilm and a biofilm that has been treated with the application of an electric current, respectively. CSnp-deposited dentin slices prevented LSCM measurement of the treated biofilm thickness due to production of streaking artefacts. However, the LSCM images show that treatment with an electric current alone may reduce the biofilm thickness. In some cases, the thickness of the biofilm may be reduced from 93 μm down to 22 μm, suggesting bacterial detachment resulting from the application of the electric current. An approximate 70% reduction in biofilm thickness was obtained after treatment with 50V (˜2.5 mA) for 10 minutes (P<0.001). Also shown in FIGS. 7A and 7B, the proportion of live cells (green) and dead cells (red) were consistent. Combination of an electric current and charged particles such as CSnp may be used to further reduce biofilm thickness and increase the ratio of dead cells to live cells.

FIG. 7C shows rendered z-axis views (i.e. depth) comparing biofilm thickness on untreated (labeled “Control”) dentin samples and dentin samples treated with an electric field (labeled “50V”) generated from the application of a 50V DC potential difference (generating approximately 2.5 mA), across the applicator electrodes for 10 minutes. A noticeable reduction in biofilm thickness was observed between the untreated and treated dentin samples. Specifically, for the present case, a statistically significant 70% reduction in biofilm was achieved (P<0.001). Table 1 summarizes the changes in the biofilm thickness at various areas of 7-week old E. faecalis biofilms grown on corresponding dentin slices shown in FIG. 7C after treatment as measured by the LSCM.

The reduction in biofilm thickness may be linked to repulsive forces between bacteria and substrate being enhanced by modification of substrate surface charge (i.e. by applying an electric field) thus provoking surface detachment of bacterial biofilms [81]. Other contributions may include, disruption of charged bacterial membranes, stimulating autolysis, electrolytic generation of oxygen, electrochemical generation of potential oxidants and the electrophoretic transport of antibacterial particles into and disruption of the charged EPS matrix by electric current [53, 56, 60].

### Mechanical Effects of Electrokinetic Delivery

Different intra-canal risk factors that may be induced by a clinician (iatrogenic), disease or age mediated (non-iatrogenic), alone or in combination, have been reported to increase the predisposition of endodontically treated teeth to fractures. Some of the non-iatrogenic factors are: bacterial colonization and the release of bacterial enzymes as well as host-derived matrix metalloproteinases leading to the degradation of hard tissue and subsequent deterioration of the mechanical properties of dentin. Some of the iatrogenic causes of compromised mechanical property in root canal treated teeth may include the chemical effects of irrigants/medicaments used during root canal treatment on dentin. For example, sodium hypochlorite (1% to 6%) and ethylenediaminetetraacetic acid (EDTA) (17%) are commonly used during root canal treatment to kill bacteria and remove debris and the smear layer. Unfortunately, these chemicals can cause physical and chemical changes in the inorganic and organic phases of the dentin, leading to adverse effects on the mechanical integrity of dentin [82]. In addition, microdefects induced by instrumentation and filling procedures may also accelerate fracture progression in dentin after root canal treatment. Currently treatment procedures capable of reversing such iatrogenic or non-iatrogenic changes in dentin do not exist.

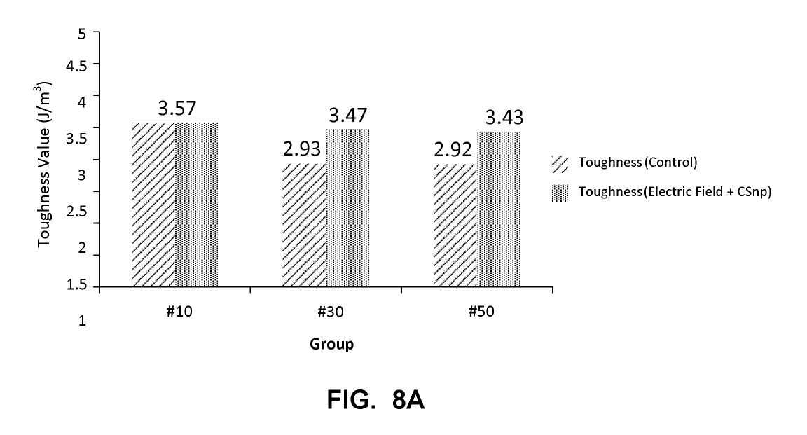

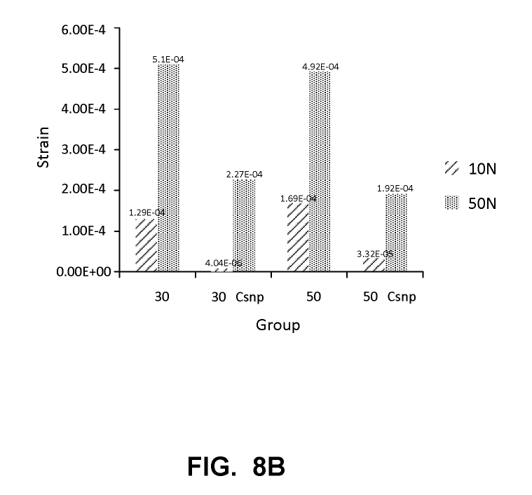

As already discussed previously, the application of an electric field alone or in combination of antimicrobial particles such as CSnp may help preserve the mechanical integrity of the tooth. In general “toughness” as expressed in J/m³ is a common mechanical property used to indicate the degree of resistance to fracture in biological hard tissues such as dentin. FIGS. 8A-8B show the results of mechanical testing that can be conducted to determine toughness of dentin, which is the total area under the stress/strain curve. FIG. 8A shows that the toughness of root dentin samples may be preserved/enhanced after conditioning with an electric field and CSnp.

FIG. 8B shows the strain distribution of root dentin samples in the direction perpendicular to dentinal tubules. Strain characteristics may be measured using digital moiré interferometry (DMI) which makes use of principles of optical interferometry to measure micro-level deformations on surfaces. It is a high-sensitive, non-destructive, real-time measurement technique that provides whole-field strain information. Moreover, DMI allows testing of in situ specimens at physiologically realistic loads [83]. Specifically the mechanical strain distribution is measured using DMI after simulated dentin removal and after conditioning the root dentin with cross-linked CSnp applied using the electrokinetic method described above. The numbers “30” and “50” indicate root canal enlargement to instrument size 30 and 50, respectively. The measurements indicate that the mechanical strain generated or deformation of samples treated with CSnp is markedly reduced.

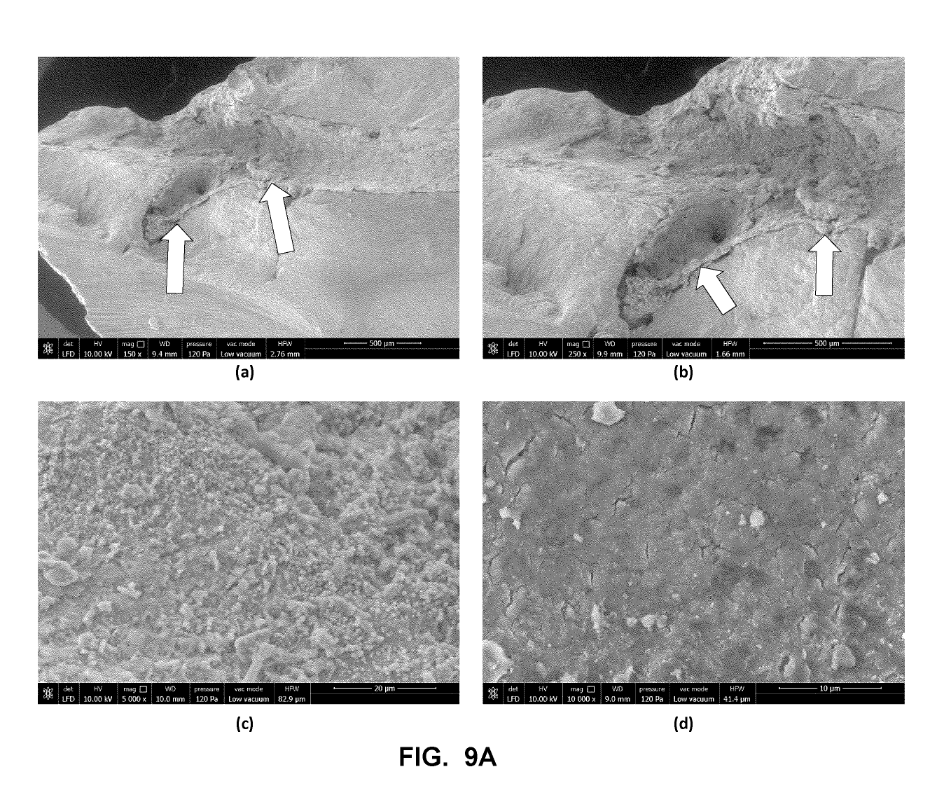

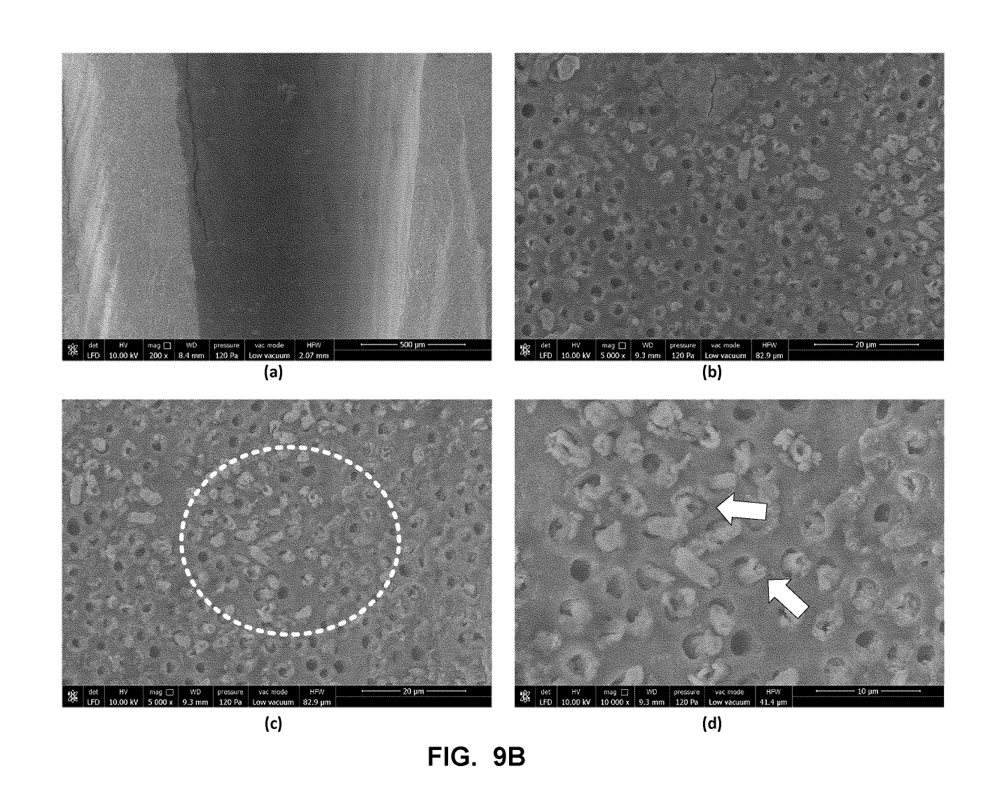

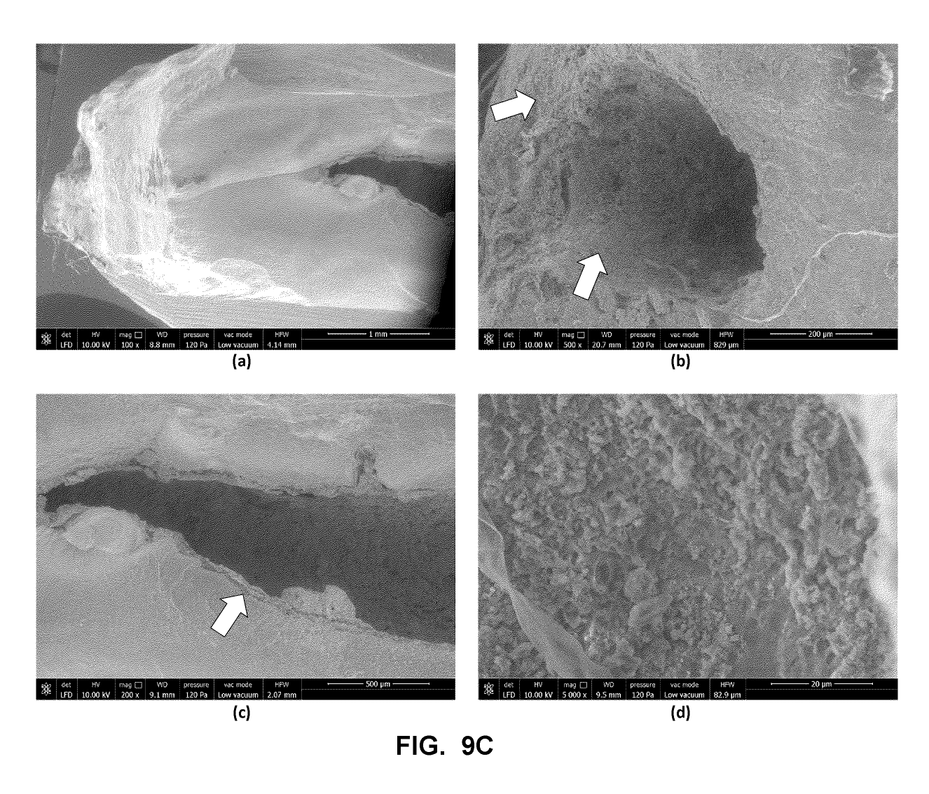

FIGS. 9A-9C show scanning electron micrographs (SEM) of surfaces of the root canal and external root surface coated with CSnp using the electrokinetic method described above. FIGS. 9A and 9B show SEM of root canal dentin surfaces coated nanoparticles. FIGS. 9A(c) and 9A(d) are high magnification views of FIGS. 9A(a) and 9A(b). FIGS. 9B(a)-(d) show SEM of a region of root canal dentin surface coated with nanoparticles at progressively higher magnification. FIG. 9B(d) is a magnified view of a region in FIG. 9B(c) that is bounded an ellipse. FIGS. 9C(a)-(d) show nanoparticles being delivered to locations beyond the root apex, and may be found as a coating on the root surface.

While the applicant's teachings described herein are in conjunction with various embodiments for illustrative purposes, it is not intended that the applicant's teachings be limited to such embodiments as these the embodiments described herein are intended to be examples. On the contrary, the applicant's teachings described and illustrated herein encompass various alternatives, modifications, and equivalents, without departing from the embodiments described herein, the general scope of which is defined in the appended claims.

TABLE 1

| | | Control^{a} | Treatment^{b}* |
|---|---|---|---|
| | | Biofilm | Biofilm |
| | | Thickness | Thickness |
| Sample | Area | (μm) | (μm) |
| 1 | 1 | 120 | 30 |
| | 2 | 130 | 30 |
| | 3 | 60 | 25 |
| | 4 | 90 | 20 |
| 2 | 1 | 60 | 15 |
| | 2 | 90 | 15 |
| | 3 | 95 | 15 |
| | 4 | 80 | 30 |
| 3 | 1 | 105 | 20 |
| | 2 | 55 | 30 |
| | 3 | 90 | 20 |
| | 4 | 140 | 15 |
| 4 | 1 | 80 | 30 |
| | 2 | 60 | 30 |
| | 3 | 100 | 20 |
| | 4 | 135 | 15 |

| | | |
|---|---|---|
| Average thickness | 93.13 | 22.50 |

^{a}untreated samples
^{b}samples received 50 V (2.0 mA) for 10 minutes
*treated samples demonstrated a statistically significant reduction (70%, P< 0.001) in thickness when compared to untreated samples

### REFERENCES

1. Nair P N R. Light and electron microscopic studies of root canal flora and periapical lesions. J Endod. 1987 January; 13(1):29-39.
2. Ricucci D, Siqueira J. Biofilms and Apical Periodontitis: Study of Prevalence and Association with Clinical and Histopathologic Findings. J Endod. 2010; 36(8):1277-88.
3. Molven O, Olsen I, Kerekes K. Scanning electron microscopy of bacteria in the apical part of root canals in permanent teeth with periapical lesions. Endod Dent Traumatol. 1991 October; 7(5):226-9.
4. Kishen A. Advanced therapeutic options for endodontic biofilms. Endod Top. 2012; 99-123.
5. Donlan R M, Costerton J W. Biofilms: Survival Mechanisms of Clinically Relevant Microorganisms. Clin Microbiol Rev. 2002; 15(2):167-93.
6. Costerton J W, Lewandowski Z, DeBeer D, Caldwell D, Korber D, James G. Biofilms, the customized microniche. J Bacteriol. 1994 April; 176(8):2137-42.
7. Sen B H, Piskin B, Demirci T. Observation of bacteria and fungi in infected root canals and dentinal tubules by SEM. Endod Dent Traumatol. 1995; 11(1):6-9.
8. Tronstad L, Barnett F, Cervone F. Periapical bacterial plaque in teeth refractory to endodontic treatment. Endod Dent Traumatol. 1990 April; 6(2):73-7.
9. Ricucci D, Siqueira J F. Apical actinomycosis as a continuum of intraradicular and extraradicular infection: case report and critical review on its involvement with treatment failure. J Endod. 2008 September; 34(9):1124-9.
10. Figdor D. Microbial aetiology of endodontic treatment failure and pathogenic properties of selected species. Aust Endod J. 2004 April; 30(1):11-4.
11. Del Fabbro M, Samaranayake L P, Lolato A, Weinstein T, Taschieri S. Analysis of the secondary endodontic lesions focusing on the extraradicular microorganisms: an overview. J Investig Clin Dent. 2013 Jun. 14;
12. Noguchi N, Noiri Y, Narimatsu M, Ebisu S. Identification and localization of extraradicular biofilm-forming bacteria associated with refractory endodontic pathogens. Appl Environ Microbiol. 2005 December; 71(12):8738-43.
13. Ricucci D, Martorano M, Bate a L, Pascon E a. Calculus-like deposit on the apical external root surface of teeth with post-treatment apical periodontitis: report of two cases. Int Endod J. 2005 April; 38(4):262-71.
14. Haapasalo M, Shen Y A, Ricucci D. Reasons for persistent and emerging post-treatment endodontic disease. Endod Top. 2011; 31-50.
15. Wang J, Chen W, Jiang Y, Liang J. Imaging of extraradicular biofilm using combined scanning electron microscopy and stereomicroscopy. Microsc Res Tech. 2013 Jul. 16; 00(May):1-5.
16. Wang J, Jiang Y, Chen W, Zhu C, Liang J. Bacterial flora and extraradicular biofilm associated with the apical segment of teeth with post-treatment apical periodontitis. J Endod. Elsevier Ltd; 2012 July; 38(7):954-9.
17. Signoretti F G C, Endo M S, Gomes BPFA, Montagner F, Tosello F B, Jacinto R C. Persistent extraradicular infection in root-filled asymptomatic human tooth: scanning electron microscopic analysis and microbial investigation after apical microsurgery. J Endod. 2011 December; 37(12):1696-700.
18. Su L, Gao Y, Yu C, Wang H, Yu Q. Surgical endodontic treatment of refractory periapical periodontitis with extraradicular biofilm. Oral Surg Oral Med Oral Pathol Oral Radiol Endod. Elsevier Inc.; 2010 July; 110(1):e40-4.
19. Siqueira J. Aetiology of root canal treatment failure: Why well-treated teeth can fail. Int Endod J. 2001; 34(1):1-10.
20. Noiri Y, Ehara A, Kawahara T, Takemura N. Participation of Bacterial Biofilms in Refractory and Chronic Periapical Periodontitis. J Endod. 2002; 28(10):679-83.
21. Gulabivala K, Patel B, Evans G, Ng Y-L. Effects of mechanical and chemical procedures on root canal surfaces. Endod Top. 2005 March; 10(1):103-22
22. Velvart, Peter, Christine I. Peters, and O. V. E. A. Peters. 2005. “Soft Tissue Management: Flap Design, Incision, Tissue Elevation, and Tissue Retraction.” Endodontic Topics (10):78-97.
23. Stropko, John J., Glen E. Doyon, and James L. Gutmann. 2005. “Root-End Management: Resection, Cavity Preparation, and Material Placement.” Endodontic Topics 11(1):131-51.
24. Ng Y-L, Mann V, Gulabivala K. Outcome of secondary root canal treatment: a systematic review of the literature. Int Endod J. 2008 December; 41(12):1026-46.
25. Ng Y-L, Mann V, Rahbaran S, Lewsey J, Gulabivala K. Outcome of primary root canal treatment: systematic review of the literature—part 1. Effects of study characteristics on probability of success. Int Endod J. 2007 December; 40(12):921-39.
26. Ng Y-L, Mann V, Rahbaran S, Lewsey J, Gulabivala K. Outcome of primary root canal treatment: systematic review of the literature—Part
2. Influence of clinical factors. Int Endod J. 2008 January; 41(1):6-31.
27. Kishen A, Shi Z, Shrestha A, Neoh K G. An investigation on the antibacterial and antibiofilm efficacy of cationic nanoparticulates for root canal disinfection. J Endod. American Association of Endodontists; 2008 December; 34(12):1515-20.
28. European Society of Endodontology. 2006. Quality Guidelines for Endodontic Treatment: Consensus Report of the European Society of Endodontology.
29. Ørstavik, D., and TR Pitt Ford. 1998. “Microbial Infection and Host Responses.” in Essential endodontology: prevention and treatment of apical periodontitis., edited by D Ørstavik and TR Pitt Ford. Oxford: Blackwell Science.
30. Shrestha A, Hamblin M R, Kishen A. Photoactivated rose bengal functionalized chitosan nanoparticles produce antibacterial/biofilm activity and stabilize dentin-collagen. Nanomedicine Nanotechnology, Biol Med. Elsevier Inc.; 2014; 10(3):491-501.
31. Reddy K M, Feris K, Bell J, Wingett D G, Hanley C, Punnoose A. Selective toxicity of zinc oxide nanoparticles to prokaryotic and eukaryotic systems. Appl Phys Lett. 2007 May 24; 90(213902):2139021-3.
32. Shrestha A, Shi Z, Neoh K G, Kishen A. Nanoparticulates for antibiofilm treatment and effect of aging on its antibacterial activity. J Endod. Elsevier Ltd; 2010 June; 36(6):1030-5.
33. Shrestha A, Kishen A. The effect of tissue inhibitors on the antibacterial activity of chitosan nanoparticles and photodynamic therapy. J Endod. Elsevier Ltd; 2012 September; 38(9):1275-8.
34. Shrestha A, Fong S-W, Khoo B-C, Kishen A. Delivery of antibacterial nanoparticles into dentinal tubules using high-intensity focused ultrasound. J Endod. Elsevier Ltd; 2009 July; 35(7):1028-33.
35. Upadya M, Shrestha A, Kishen A. Role of efflux pump inhibitors on the antibiofilm efficacy of calcium hydroxide, chitosan nanoparticles, and light-activated disinfection. J Endod. Elsevier Ltd; 2011 October; 37(10): 1422-6.
36. Ohl S-W, Shrestha a, Khoo B C, Kishen a. Characterizing bubble dynamics created by high-intensity focused ultrasound for the delivery of antibacterial nanoparticles into a dental hard tissue. Proc Inst Mech Eng Part H J Eng Med. 2010 Nov. 1; 224(11):1285-96.
37. Jung B-O, Kim C-H, Choi K-S, Lee Y M, Kim J-J. Preparation of amphiphilic chitosan and their antimicrobial activities. J Appl Polym Sci. 1999 Jun. 24; 72(13):1713-9.
38. Rabea El, Badawy M E-T, Stevens C V, Smagghe G, Steurbaut W. Chitosan as antimicrobial agent: applications and mode of action. Biomacromolecules. 2003; 4(6):1457-65.
39. Park E, Shen Y A, Haapasalo M. Irrigation of the apical root canal. Endod Top. 2013; (6):54-73.
40. Hamaker H C, Verwey E J W. Part II.—(C) Colloid stability. The role of the forces between the particles in electrodeposition and other phenomena. Trans Faraday Soc. The Royal Society of Chemistry; 1940; 35(0):180-5.
41. Pickard W F. Remarks on the Theory of Electrophoretic Deposition. J Electrochem Soc. 1968 Apr. 1; 115(4):105C-108C.
42. Del Pozo J L, Rouse M S, Mandrekar J N, Steckelberg J M, Patel R. The electricidal effect: reduction of Staphylococcus and pseudomonas biofilms by prolonged exposure to low-intensity electrical current. Antimicrob Agents Chemother. 2009 January; 53(1):41-5.
43. Boccaccini A R, Keim S, Ma R, Li Y, Zhitomirsky I. Electrophoretic deposition of biomaterials. 2010; (May).
44. Pang X, Casagrande T, Zhitomirsky I. Electrophoretic deposition of hydroxyapatite—CaSiO3—chitosan composite coatings. J Colloid Interface Sci. Elsevier Inc.; 2009; 330:323-9.
45. Probstein R F. Physicochemical Hydrodynamics. 2nd editio. Physicochemical Hydrodynamics. Wiley; 1994.
46. Hunter R J. Zeta Potential in Colloid Science: Principles and Applications. Academic Press; 1981.
47. Hamaker H C. Formation of deposit by electrophoresis. Trans Faraday Soc. 1940; 36:279-87.
48. Besra L, Liu M. A review on fundamentals and applications of electrophoretic deposition (EPD). Progress in Materials Science. 2007. p. 1-61.
49. Basu R N, Randall C A, Mayo M J. Fabrication of Dense Zirconia Electrolyte Films for Tubular Solid Oxide Fuel Cells by Electrophoretic Deposition. J Am Ceram Soc. 2001; 84(1):33-40.
50. Wang Y-C, Leu I-C, Hon M-H. Kinetics of Electrophoretic Deposition for Nanocrystalline Zinc Oxide Coatings. J Am Ceram Soc. 2004; 87(1):84-8.
51. Chen F, Liu M. Preparation of yttria-stabilized zirconia (YSZ) films on La 0.85Sr 0.15MnO 3 (LSM) and LSM-YSZ substrates using an electrophoretic deposition (EPD) process. J Eur Ceram Soc. 2001; 21(2):127-34.
52. Zhitomirsky I, Gal-Or L. Electrophoretic deposition of hydroxyapatite. J Mater Sci Mater Med. 1997; 8:213-9.
53. Freebairn D, Linton D, Harkin-Jones E, Jones D S, Gilmore B F, Gorman S P. Electrical methods of controlling bacterial adhesion and biofilm on device surfaces. Expert Rev Med Devices. 2013 January; 10(1):85-103.
54. Anderson A K, Finkelstein R. A study of the electropure process of treating milk. J Dairy Sci. 1919; 2:374-406.
55. Del Pozo J L, Rouse M S, Euba G, Kang C-I, Mandrekar J N, Steckelberg J M, et al. The electricidal effect is active in an experimental model of Staphylococcus epidermidis chronic foreign body osteomyelitis. Antimicrob Agents Chemother. 2009 October; 53(10):4064-8.
56. Del Pozo J L, Rouse M S, Patel R. Bioelectric effect and bacterial biofilms. A systematic review. Int J Artif Organs. 2008 September; 31(9):786-95.
57. Nichols W W, Evans M J, Slack M P, Walmsley H L. The penetration of antibiotics into aggregates of mucoid and non-mucoid Pseudomonas aeruginosa. J Gen Microbiol. 1989; 135:1291-303.
58. Gilbert P, Collier P J, Brown M R. Influence of growth rate on susceptibility to antimicrobial agents: biofilms, cell cycle, dormancy, and stringent response. Antimicrob Agents Chemother. 1990; 34(10):1865-8.
59. Eng R H K, Padberg F T, Smith S M, Tan E N, Cherubin C E. Bactericidal Effects of Antibiotics. Antimicrob Agents Chemother. 1991; 35(9):1824-8.
60. Zhang J, Neoh K G, Hu X, Kang E-T. Mechanistic insights into response of Staphylococcus aureus to bioelectric effect on polypyrrole/chitosan film. Biomaterials. Elsevier Ltd; 2014 September; 35(27):7690-8.
61. Van der Borden a J, van der Mei H C, Busscher H J. Electric block current induced detachment from surgical stainless steel and decreased viability of Staphylococcus epidermidis. Biomaterials. 2005 November; 26(33):6731-5.
62. Kronfeld R. Histologic study of the influence of function on the human periodontal membrane. J Am Dent Assoc. 1931; 18(1242).
63. Fernandez D P, Mulev Y, Goodwin A, Levelt Sengers J. A Database for the Static Dielectric Constant of Water and Steam. J Phys Chem Ref Data. 1995;
64. Jo B, Van Lerberghe L M, Motsegood K M, Beebe D J. Three-Dimensional Micro-Channel Fabrication in Polydimethylsiloxane (PDMS) Elastomer. J Microelectromechanical Syst. 2000; 9(1):76-81.
65. Whitesides G M, Ostuni E, Takayama S, Jiang X, Ingber D E. Soft Lithography in Biology and Biochemistry. Annu Rev Biomed Eng. 2001; 3:335-73.
66. Huang M, Ma Z, Khor E, Lim L Y. Uptake of FITC-chitosan nanoparticles by A549 cells. Pharm Res. 2002; 19(10):1488-94.
67. Ma Z, Lim L-Y. Uptake of Chitosan and Associated Insulin in Caco-2 Cell Monolayers: A Compar . . . Pharm Res. 2003; 20(11):1812-9.
68. Dow Corning 200 Fluid, Information about Dow Corning Silicone Fluid. Midland, Mich.: Dow Corning Corp.; Form No. 22-931A-90, 22-926D-93, 2 p.
69. Conlisk A T. The Debye-Hückel approximation: Its use in describing electroosmotic flow in micro- and nanochannels. Electrophoresis. 2005; 26(10):1896-912.
70. Pfeiffer E a. Electrical stimulation of sensory nerves with skin electrodes for research, diagnosis, communication and behavioral conditioning: A survey. Med Biol Eng. 1968; 6(6):637-51.
71. Wilson B L, Broberg C, Baumgartner J C, Harris C, Kron J. Safety of Electronic Apex Locators and Pulp Testers in Patients With Implanted Cardiac Pacemakers or Cardioverter/Defibrillators. J Endod. 2006; 32(9):847-52.
72. Meredith N, Gulabivala K. Electrical impedance measurements of root canal length. 1997; 44(0):126-31.
73. Dalziel C F. The Threshold of Perception Currents. Trans Am Inst Electr Eng Part III, Power Appar Syst. 1954; 73(2):990-6.
74. Martinsen O G, Grimnes S, Piltan H. Cutaneous perception of electrical direct current. Itbm-Rbm. 2004 October; 25(4):240-3.
75. Poortinga a T, Bos R, Busscher H J. Controlled electrophoretic deposition of bacteria to surfaces for the design of biofilms. Biotechnol Bioeng. 2000 Jan. 5; 67(1):117-20.
76. Tsong T Y. Electrical Modulation of Membrane Proteins. Annu Rev Biophys Biophys Chem. 1990; 19:83-106.
77. Calvin N M, Hanawalt P C. High-efficiency transformation of bacterial cells by electroporation. J Bacteriol. 1988; 170(6):2796-801.
78. Shimada K, Shimahara S. Leakage of Cellular Contents and Morphological Changes in Resting Escherichia coli B Cells Exposed to an Alternating Current. Agric Biol Chem. 1895; 49(12):3605-7.
79. Costerton J W, Ellis B, Lam K, Johnson F, Khoury a. E. Mechanism of electrical enhancement of efficacy of antibiotics in killing biofilm bacteria. Antimicrob Agents Chemother. 1994; 38(12):2803-9.
80. Katsikogianni M, Missirlis Y F. Concise review of mechanisms of bacterial adhesion to biomaterials and of techniques used in estimating bacteria-material interactions. Eur Cells Mater. 2004; 8:37-57.
81. Ueshima M, Tanaka S, Nakamura S, Yamashita K. Manipulation of bacterial adhesion and proliferation by surface charges of electrically polarized hydroxyapatite. J Biomed Mater Res. 2002; 60(4):578-84.
82. Kishen A, Mechanisms and risk factors for fracture predilection in endodontically treated teeth. Endodontic Topics. 2006; 13: 57-83.
83. Kishen A, and Asundi, A., Investigations of thermal property gradients in the human dentine. J. Biomed. Mater. Res. 2001; 55: 121-130.

1. A method of reducing bacteria of a tooth receiving root canal treatment, the tooth having a root canal, wherein the method comprises:
deploying an intracanal electrode within the root canal;
deploying an intraoral electrode in close proximity to the root canal and external to the tooth; and
applying a potential difference between the electrodes to create an electric field therebetween to reduce the bacteria of the tooth.

2. The method of claim 1, wherein the method further comprises introducing a carrier solution having charged particles into the root canal and applying the potential difference to create the electric field to induce a flow of the charged particles to structures within and adjacent to the tooth.

3. The method of claim 1, wherein the act of deploying the intraoral electrode comprises placing the intraoral electrode in direct contact with soft tissue surrounding the tooth, placing the intraoral electrode in direct contact with tooth enamel or placing the intraoral electrode in direct contact with the periodontal ligament.

4. The method of claim 1, wherein the intraoral electrode comprises a conductive ring and the method comprises placing the intraoral electrode in contact with one or more areas of oral soft tissue adjacent to the tooth.

5. The method of claim 1, wherein the intraoral electrode comprises a microneedle array electrode and the method comprises deploying the microneedle electrode on soft oral tissue adjacent to the tooth.

6. The method of claim 1, wherein the intraoral electrode comprises a modified conductive rubber dam clamp and the method comprising placing the modified rubber dam clamp adjacent to or penetrating into the gingival tissues on a buccal and a lingual region of the tooth.

7. The method of claim 1, wherein the method comprises applying a potential difference between the electrodes of about 20V to 70V AC or DC.

8. The method of claim 1, wherein the method comprises applying the potential difference between the electrodes to provide the electric field with a field strength of about 15 V/cm to about 45 V/cm.

9. The method of claim 1, wherein the method further comprises delivering the charged particles to locations corresponding to at least one of an external root surface, a periapical region, and a cementum surface of the root canal.

10. The method of claim 9, wherein the method further comprises applying the electric field to form a coating of particle film using at least some of the particles, the coating of particle film being formed on at least one of the external root surface, the periapical region, and the cementum surface of the root canal.

11. The method of claim 1, wherein
the method comprises applying the electric field to accumulate at least some of the charged particles in a region of the tooth corresponding to the apical delta.

12. The method of claim 1, wherein the method comprises applying the electric field to form a film of at least some of the charged particles on an extraradicular root surface of the tooth.

13. The method of claim 1, wherein the method further comprises providing the carrier solution with charged particles having at least one of a sufficiently low conductivity of about 5-500 μS/m, a low viscosity of about 0.5-5 cP and a high dielectric constant of about 50-80 to facilitate movement and deposition of charged particles within the tooth.

14. The method of claim 1, wherein the charged particles comprise nanoparticles with a diameter less than 1 μm and belong to a class of particles that are sensitive to the application of an electric field.

15. The method of claim 1, wherein the charged particles comprise chitosan nanoparticles.

16. A system for reducing bacteria of a tooth receiving root canal treatment, the tooth having a root canal, wherein the system comprises:
an intracanal electrode that is deployed within the root canal;
an intraoral electrode that is deployed in close proximity to the tooth canal but external of the tooth; and
a signal generator coupled to the electrodes for applying a potential difference between the electrodes to create an electric field therebetween to reduce the bacteria of the tooth.

17. The system of claim 16, wherein the system further comprises a carrier solution having charged particles and the charged particles are introduced into the root canal before or during application of the electric field wherein the electric field is created to induce a flow of the charged particles to structures within and adjacent to the tooth.

18. The system of claim 16, wherein the system further comprises a power supply that is coupled to the signal generator and is configured to maintain at least one of a constant voltage and a constant current output.

19. The system of claim 16, wherein the intracanal electrode comprises a modified conducting endodontic file having an electrically conducting shaft and an insulated tip that is configured to laterally drive the charged particles towards dentin of the tooth to create an Electrophoretic Deposition (EPD) on the dentinal walls of the tooth during use.

20. The system of claim 19, wherein a portion of the shaft is insulated to prevent aberrant currents in cervical portions of the tooth and adjacent teeth.

21. The system of claim 16, wherein the intracanal electrode comprises a modified conducting endodontic file have an electrically insulated shaft and a conducting tip to generate electric field lines through an apical foramen or other ramifications of the tooth during use.

22. The system of claim 16, wherein the intracanal electrode comprises a modified conductive ultrasonic tip, at least a portion of the intracanal electrode is covered by an insulating sheath and ultrasonic energy is delivered before, during or after application of the electric field.

23. The system of claim 16, wherein the intracanal electrode comprises a hollow needle that is coupled to a fluid reservoir for providing the carrier solution with the charged particles during use, at least a portion of the needle is insulated to direct the electrical field directed toward lateral or apical aspects of the root canal.

24. The system of claim 23, wherein the needle may be open-ended, side-beveled, or side-vented for delivering and replenishing the carrier solution into the root canal simultaneously or subsequently to delivering electric current within the root canal.

25. The system of claim 16, wherein the intracanal electrode is connectable to a dental ultrasonic unit.

26. The system of claim 16, wherein the intraoral electrode is configured and deployed to make direct contact with one of soft tissue surrounding the tooth, tooth enamel, and a periodontal ligament of the tooth during use.

27. The system of claim 16, wherein the intraoral electrode comprises a conductive ring that is configured to be placed around the tooth and make contact with one or more areas of soft oral tissue around the tooth during use.

28. The system of claim 16, wherein the intraoral electrode comprises a microneedle array electrode that is configured for deployment on the soft oral tissue adjacent the tooth during use.

29. The system of claim 16, wherein the intraoral electrode comprises a modified rubber dam clamp that is configured to come into contact or penetrate gingival tissues on a buccal region and a lingual region of the tooth.

30. The system of claim 16, wherein the potential difference generated by the signal generator between the electrodes is about 20V to about 70V AC or DC.

31. The system of claim 16, wherein the potential generated by the signal generator produces the electric field between the electrodes with an electric field strength from about 15 V/cm to about 45 V/cm.

32. The system of claim 16, wherein the carrier solution with charged particles has at least one of a sufficiently low conductivity of about 5-500 μS/m, a low viscosity of about 0.5-5 cP and a high dielectric constant of about 50-80 to facilitate movement and deposition of charged particles within the tooth during use.

33. The system of claim 16, wherein the charged particles comprise nanoparticles with a diameter less than 1 μm and are sensitive to the application of an electric field.

34. The system of claim 16, wherein the charged particles comprise chitosan nanoparticles.
